(19) United States
(12) Patent Application Publication
(10) Pub. No.: US 2025/0275705 A1
(43) Pub. Date: Sep. 4, 2025

(54) DIAGNOSTIC SYSTEM

(71) Applicant: Falko SKRABAL, Graz (AT)

(72) Inventors: Falko SKRABAL, Graz (AT); Katharina SKRABAL, Graz (AT); Philipp GLITZNER, Wettmannstetten (AT)

(73) Assignee: Falko SKRABAL, Graz (AT)

(21) Appl. No.: 18/861,081

(22) PCT Filed: Apr. 28, 2023
(86) PCT No.: PCT/AT2023/060146

§ 371 Date: Oct. 28, 2024

(30) Foreign Application Priority Data
May 3, 2022 (AT) A 50305/2022

(51) Int. Cl. A61B 5/282; A61B 5/00; A61B 5/0205; A61B 5/022; A61B 5/0295; A61B 5/097; A61B 5/1455; A61B 5/26; A61B 5/352; A61B 7/00

(52) U.S. Cl. CPC A61B 5/282; A61B 5/0205; A61B 5/26; A61B 5/352; A61B 5/4836; A61B 5/6831; A61B 5/6843; A61B 5/6891; A61B 5/6892; A61B 5/0077; A61B 5/022; A61B 5/0295; A61B 5/097; A61B 5/14551; A61B 7/003; A61B 2562/164; A61B 2562/168

(57) ABSTRACT

A diagnostic system includes one or more support surfaces, on which a person to be examined can be positioned. To be able to make a diagnosis without specialist staff, electrodes are provided to be arranged on various regions of the support surface, particular on leg regions, on arm regions, on an upper body region and/or on a neck region. These electrodes are at least partly embodied as electrode pairs, wherein each electrode pair has a feed electrode and/or a measurement electrode. The feed electrode is arranged distally relative to the measurement electrode. A device is provided for feeding an electric current between the feed electrodes and the diagnostic system is configured to measure a voltage drop between the measurement electrodes in order to be able to determine impedances of body regions of a person located on the diagnostic system.

The invention relates to a diagnostic system, comprising one or more support surfaces, on which a person to be examined can be positioned, in particular seated or supine position.

Devices of this type have become known from the prior art, for example, to create an electrocardiogram (ECG) of a person to be examined. In particular, document AT 514017 B1 discloses an ECG device with chest electrodes and extremity electrodes, in which, in addition to chest electrodes and extremity electrodes, at least one further electrode is attached to the body, which is suitable for feeding and/or measuring an alternating current.

At the same time, the health awareness of people has also changed significantly. People want to be precisely informed about their health status, which is why the self-monitoring systems, such as heart rate measurements, self-recording of the ECG, heart rate or oxygen saturation, have recently become widespread.

Examples of non-invasive diagnostic methods with sensors attached to the body include temperature measurement, thermography, oxygen measurement, blood pressure measurement, ECG, whole-body or segmental impedance measurement and whole-body or segmental impedance plethysmography.

The ECG provides information about the spread of electrical cardiac excitation and any deviations, whereby it is only possible to provide a reliable diagnosis, if all relevant leads, e.g. also the Wilson leads, are included. Therefore, diagnostic systems that do not include the leads according to Einthoven, Goldberger and Wilson are essentially unsuitable for cardiovascular diagnostics and it has not yet been possible to derive the chest wall leads from electrodes attached to the back.

Whole-body impedance with electrodes on the soles of the feet only provides insufficiently accurate results about the overall body composition and the composition of the individual body segments: thorax, abdomen, left arm, right arm, left leg, right leg.

Segmental impedance measurement, on the other hand, provides precise results about body composition, fluid content, extracellular volume, edema, overhydration, muscle mass and fat mass, or fat distribution in the body. When measuring impedance, it is important that the electrodes are attached to the physiologically relevant positions of the living being so that all segments that contribute to the quantity of the measured substance are measured and unnecessary, distorting series resistances are excluded. These include, in particular, the feet and hands, because they consist essentially of connective tissues such as tendons, fascia and bones and cause high and variable resistance. The whole-body impedance, even without separate consideration of the chest and abdomen and the four extremities, also produces only unsatisfactory results.

Segmental impedance plethysmography also provides results on the condition of cardiac output, such as fiber tension of the heart muscle fibers, detection of heart failure, blood flow to the extremities or on the pulse wave duration and thus on the health of the aorta.

A disadvantage of diagnostic systems of the type mentioned at the beginning is that personnel are required to examine a person, in particular to attach the individual electrodes to the respective body regions.

This is where the invention comes into play. The object of the invention is to provide a diagnostic system for creating diagnoses and an electrocardiogram, which eliminates the above shortcomings, can be operated particularly easily without any personnel, especially medical personnel, and can still provide detailed and correct diagnoses.

This object is achieved according to the invention by a diagnostic system of the type mentioned at the outset, in which different areas of the support surfaces, in particular Electrodes are arranged on leg areas, on arm areas, on an upper body area and/or on a neck area, these being at least partially designed as pairs of electrodes, each pair of electrodes having a feed electrode and/or a measuring electrode, the feed electrode being arranged distally relative to the measuring electrode, and wherein a device for feeding an electrical current between the supply electrodes is present and wherein the diagnostic system is set up to measure a voltage drop between the measuring electrodes in order to be able to determine impedances of body areas of a person located on the diagnostic system.

Preferred variants of the embodiment are the subject of the dependent claims.

Electrodes through which an electrical current can be fed in are considered feed electrodes. Electrodes between which a voltage drop can be measured are considered measuring electrodes.

It is preferably provided that the diagnostic system is set up to switch individual electrodes or pairs of electrodes in pairs in such a way that a current, in particular an alternating current, is fed between two supply electrodes and a voltage drop between the assigned measuring electrodes can be measured in order to determine impedances of body areas the person in the diagnostic system.

In the diagnostic system according to the invention, a manual attachment of the electrodes to the respective body regions by skilled personnel is no longer required, but the support surface, which is in contact with the person to be examined when a person to be examined is positioned on the diagnostic system, is provided with electrodes and sensors that a corresponding electrocardiogram and a complete body and health analysis can be created simply by positioning a person correctly on the diagnostic system. During use, the electrodes and sensors can be in direct contact with the skin. Alternatively, however, it is also possible to carry out the measurement through a garment.

The preferred arrangement of two measuring electrodes between two feed electrodes, with an electrical current being applied via the feed electrodes, has the particular advantage that a representative voltage drop, based on which the impedance of a body region can be determined, can only be measured with a certain distance between the electrodes, through which a current is introduced into the body region. The background is that a current flow from the electrode spreads out in roughly a conical shape in the body, so that a close area around the electrode would have a higher resistance or a higher impedance due to the propagation of the current flow in the body. If a voltage drop is only detected at a certain distance from the feed electrodes, this error is thus easily by using the measuring electrodes that are arranged between the feed electrodes.

The measuring electrodes can be spaced 1 cm to 20 cm apart from the feed electrodes, preferably 2 cm to 6 cm.

With the diagnostic system according to the invention, it is thus possible to record physical properties and electrical resistances of body regions of a person positioned on the diagnostic system, it is possible to determine a health status based on the results of the resistances and sensor measurements.

This is especially possible when capacitive electrodes are used (see, for example, Lopez A et al, “Capacitive electrocardiographic and bioelectric electrodes.” IEEE Transactions on Biomedical Engineering, no. 1, pp. 99-99, 1969), which are also in flexible shape (Shuting Liu S et al, Flexible Non-contact Electrodes for Bioelectrical Signal Monitoring, Annual Int. Conf. IEEE Eng Med Biol Soc. 2018 July/2018:4305-4308). The impedance measurements and their subcomponents can also be carried out using capacitive measurements (Swegundo A et al, Sensors 2019, 19, 2539). Impedance tomographic methods can also be realized in this way, especially if the number of contactless electrodes is increased and placed strategically, for example in a circular manner on the chest strap (Yandan Jiang et al, 2019 Meas. Sei. Technol. 30 114001).

In order to be able to determine the impedances of the individual body regions, individual feed electrodes or measuring electrodes can be activated sequentially in pairs by switching off electrodes that are not required using switching devices, usually as close as possible to the support surface, namely the contact or coupling point of the person to be examined or of a patient to the electrode on the support surface or to the electrodes of the chest strap that may be used. Alternatively or additionally, two or more power sources can be provided, which are preferably galvanically isolated from one another and can be connected to the supply electrodes. The power sources could be alternating or direct current sources depending on the intended application.

To determine impedances of body areas at different frequencies, it is advantageous if the diagnostic system is set up to feed currents through the individual feed electrodes with different frequencies, in particular with frequencies of 1 Hz to 1 MHz, in particular 5 KHz to 500 KHz. The alternating current resistance or apparent resistance or phase angle of individual body regions at different frequencies can allow precise statements to be made about diseases in the respective body region. Corresponding correlations can also be created in a simple manner with the diagnostic system according to the invention, for example by determining diagnoses using methods known from the prior art, in particular by magnetic resonance tomography, by computer tomography, whole-body DXA, ultrasound and the like, with the sensor values, Impedance values and the subcomponents of the impedance, real part, imaginary part, phase angle are compared, which were determined with the diagnostic system according to the invention. This allows an initial and then continuously improving calibration of the diagnostic system to be carried out, for example using methods of artificial intelligence such as neural networks, especially “deep convolutional neural nets” for this purpose.

In principle, a corresponding diagnosis could be carried out using several electrodes at the same time. In order to achieve a particularly high level of accuracy, switches are preferably provided with which the individual electrodes can be switched directly adjacent to the support surface or at the coupling point adjacent to the person. It is then preferred that all electrodes, except for two pairs of electrodes, are connected directly the support surface, preferably less than 10 cm away from the support surface, so that an impedance between the two active electrodes can be determined without being influenced by parasitic electrical effects of the other electrodes Accordingly, switches for the electrodes are usually arranged directly on the support surface.

For example, in a corresponding method, a pair of electrodes in a left forearm area and a pair of electrodes in an upper body area can first be activated and all other electrodes can be switched off directly on the support surface in order to capture an impedance at different frequencies in the left arm by applying an alternating electrical current with different frequencies. In a next step, electrodes in the left lower leg area and in the upper body area, for example, can then be activated. In order to detect an impedance in the left leg, the voltage drop between the left and right legs must then be measured, with all other electrodes immediately on the support surface being switched off. The part of the body not traversed by the current, in this case the right leg, would then only be used as an electrical conductor. As an alternative to detecting the impedance of the left leg, the current could also be fed between the left and right lower leg and the voltage drop between the upper body or arms and the left leg could be measured. In this case, the upper body is not exposed to the current and serves as an electrical conductor. In a corresponding manner, impedances of the body to be examined are determined in an analogous manner at different frequencies for the individual body areas or body segments, for example arms, legs, lower legs, chest and abdomen. It is usually also beneficial to record side differences between left and right extremities, as asymmetries are relevant, for example in edema or muscle mass. An electrical resistance or impedance network of the body of the person to be examined can be determined, based on which, for example, a body fat percentage, a muscle mass, a bone density, an intracellular and extracellular water percentage, and the like can be determined. In this resistance network, only the part of the body through which the alternating current flows is measured; the parts of the body that are not passed through by the alternating current can be measured electrical conductors are used, and this body part is then not part of the measured body segment.

It is advantageous if an electrode contact measurement is provided, with which those electrodes can be identified which have the lowest contact resistance to a person to be examined, the diagnostic system being set up to activate those electrodes which have the lowest contact resistance, in particular by a switch near the coupling point. This means that those electrodes that have the lowest contact resistance to the body of the person to be examined can be used automatically for a diagnosis, i.e. for which electrodes electrical contact is best in order to exclude measurement errors as much as possible. Thus, for example, electrodes may be arranged distributed over the entire diagnostic system, wherein for a measurement, those electrodes can be activated in which the contact is best and thus the measurement result is most meaningful. It goes without saying that individual electrodes can be used both as measuring electrodes and as feed electrodes, with a current being impressed into the person via the feed electrodes and a voltage drop being measured between the measuring electrodes.

It is advantageous if an electrode contact measurement is configured to apply an electrical voltage to individual electrodes in order to achieve a current flow via a body located on the diagnostic system, so that a contact resistance can be determined on the basis of the level of the current. Therefore, it is possible to determine a contact quality based on the contact resistance.

In order to avoid measurement errors during the examination, it is preferably provided that the electrode contact measurement can be switched off, in particular close to the coupling point to the person.

Alternatively, it is also provided that current sources matched with the feed electrodes, which are placed, connected or connectable in or near the electrodes, which are, in particular, synchronous.

Instead of using precisely matched power sources, the power sources can also be regulated taking into account the person's common-mode voltage, which is measured, for example, via the right-leg drive electrode. It is preferably provided that the impedance measurements are carried out as a pulse-synchronous plethysmographic measurement and as a multi-frequency impedance measurement From this, an impedance spectroscopy and a Cole-Cole plot can be produced with few measured frequencies. In particular, it is also planned to carry out the segmental pulse-synchronous impedance plethysmographic measurements, i.e. the change in the volume of a segment with the heartbeat, in such a way that part of the measuring path is not traversed by the measuring current. For example, a measurement of a leg plethysmogram or a lower leg plethysmogram can be designed in such a way that the measuring current is fed between the neck and leg or between the arm and leg, but the measurement of the leg plethysmogram can be carried out between the two measuring electrodes on the lower leg, with the contralateral leg not through which the current flows only serves as an electrical conductor. This means that only the plethysmogram can be measured, not of the entire leg, but only of the lower leg, if the leg through which the current flows is measured between the knee electrode and the lower leg electrode or between the knee electrode and the leg electrode. This also applies to the arm and chest plethysmograms. To obtain an arm plethysmogram, for example, the current could be fed between the two arms, or between an arm and a leg; the measurement would then be carried out either between a leg (not through which the current flows) and the arm through which the current flows, or otherwise between both arms then one arm does not have a current flowing through it. For the thoracic plethysmogram, for example, the input could be between an arm and a leg and between the neck electrode (or forehead electrode) and one of the chest wall ECG electrodes, or neck or forehead electrode and an electrode attached to the support surface in the torso area. The chest wall electrodes or the electrodes attached to the support surface in the torso area can also be used together with one of the extremity electrodes to supply electricity.

The plethysmograms are preferably arranged from several to many heartbeats and so-called templates are constructed so that the exact characteristics of the templates such as slope, steepness, peak height drop slope, and a shape analysis can then be used to create diagnoses, e.g. for measuring cardiac output, detection of heart failure, volume wave transit time, blood flow differences of left and right extremities. Methods of artificial intelligence, especially neural networks and “deep convolutional neural networks” have proven particularly useful for the shape analysis of the templates.

It has been proven useful to have a multi-lead ECG.

In order to be able to reconstruct the conventional ECG leads, in particular Einthoven I, II, III, Wilson V1-V6 and Goldberger leads aVR, aVL, aVF, from the electrode positions available on the support surface, the leads should ideally be in the three-dimensional space of the torso be arranged on the orthogonal x, y, z axes to accurately determine the cardiac vectors and reconstruct the conventional leads. For example, the EASI leads according to Dower (J Electrocardiology, Suppl 1988, S182-S187) have proven useful in the past, although they require electrodes on the front of the chest.

In the current application we are not looking for similarities between the back leads and the conventional ECG leads, but rather reconstruct the conventional leads based on physical principles from strategically optimally arranged electrode positions on the support surface. This is then made possible by vector cardiography according to Frank (Circulation, 13, 737-49, 1956), with the help of which the classic derivations according to Wison, Einthoven and Goldberger can be reconstructed. For this purpose, at least three ECG leads should be arranged on the support surface, if possible in all three levels of the room and as orthogonally as possible in relation to the person positioned on the support surface: one lead approximately centrally and vertically, approximately corresponding to the longitudinal axis of the support surface (and thus also the longitudinal axis of the human body) in the upper region of the support surface, the second lead approximately in the central longitudinal region of the support surface approximately transverse to the longitudinal direction of the support surface (and thus also corresponding to the horizontal axis of the human body) and the third lead approximately diagonally in the upper region of the support surface and thus corresponding to the sagittal axis of the human body lying on the support surface. Ideally, this sagittal axis should be directed at approximately 10:30, corresponding to an hour hand on the clock, or vice versa at 4:30. The three-dimensionality arises because the base of the heart is in the middle of the chest, while the tip of the heart is on the front wall of the chest. This can then be used in analogy to the derivations from Dower (J Electrocardiology 21, Supplement, 1988, S182-S187) to reconstruct the conventional leads, especially according to Wilson. Reference is made to the corresponding literature cited here.

Alternatively, the hour hand could also be pointed at 1 hour 30 so that the third lead corresponds to the sagittal axis with the person lying on the support surface.

On the support surface, these leads could be arranged with the electrode positions “Iᵣ”, “Mᵣ”, “Aᵣ” in a plane at the level of approximately the 10th to 12th vertebral body, which is approximately the 5th intercostal space at the level of the sternum. Another electrode can be placed on the neck, for example, “Nᵣ”. As we found out, due to the tilted position of the heart in three-dimensional space, these electrode positions are located in such a way that a reconstruction of the standard leads according to Einthoven, Goldberger and Wilson is possible.

Since these leads are located on the back of the person, the coefficients here Iᵣ, Mᵣ, Aᵣ are each referred to “rear” and Nᵣ for neck rear. Due to the simplified position of the electrodes, they no longer correspond exactly to the position of the Frank network with xyz signals, but rather linear derivatives of these x′y′z′. In order to return to the original xyz positions according to Frank (Circulation, 13, 737-49, 1956), in addition to the correction using software, an analog correction box could also be connected to the ECG device. In addition, the limb electrodes with leads I, II, III, avR, avL and avF can also be used to reconstruct the missing conventional 12-channel ECG leads in order to additionally reconstruct the missing leads, since these leads are connected by the feed ECG signals that can be obtained using measuring electrodes on the arms and legs can be available anyway. Similar to the method of e.g. Dower or e.g. Xue et al. (Computers In Cardiology, IEEE, 2007; 709-71), for example, the chest wall leads can be reconstructed.

$$V derived = a ( {A}_{r} - {I}_{r} ) + b ( {M}_{r} - {N}_{r} ) + c ( {A}_{r} - {N}_{r} ) + d$$

Derivative Aᵣ to Iᵣ would represent the horizontal vector component, Mᵣ to Nᵣ the vertical vector component and Aᵣ to Nᵣ the anterior-posterior vector component. The anterior-posterior component could be strengthened by placing the neck electrode more ventrally in a U-shaped neck roll that partially encloses the neck, or by placing the neck electrode ventrally in the area of the larynx, for example on a larynx microphone Sternum, or on the forehead as a forehead electrode 45, for example in a headband, or for example in the support surface of virtual reality glasses, for example on the forehead (Fr for front) Then the equations could be

$$V derived = a ( {A}_{r} - {I}_{r} ) + b ( {M}_{r} - {N}_{r} ) + c ( {A}_{r} - Fr ) + d$$

Such equations are created for the leads V1 to V6, and if necessary also for the Einthoven and Goldberger derivatives, in order to reconstruct the other leads using the respective coefficients. With the help of multiple, for example linear regression equations using the amplitudes of individual segments of the ECG curve, namely the PR segment, QRS complex, ST-T segment and the T wave, the conventional derivations can be reconstructed even better, especially if for each of the Separate sets of equations are developed for the above sections of the ECG curve, taking the root mean square error into account. These Dower derivations have proven very useful in clinical practice as a replacement for the conventional Einthoven, Goldberger and Wilson derivations (Klein N D, Computers in Cardiology 1997, 24:721-24), and we propose an analogous approach here with the new positions of the electrodes on the support surface. A patient-specific segment-specific (PSSS) algorithm provides a particularly precise determination of the leads V1, V2, V3, V4, V5 and V6 (Scherer J A, JACC 15: 191A, 1990) and can be used here for the new leads. Thus, an orthogonal set of ECG leads derived from our new ECG leads V derived=a (Aᵣ−Iᵣ)+b(Mᵣ−Nᵣ)+c(Aᵣ−Nᵣ)+d can also be derived with the help of the Levkov transformation (Levkov, Med Biol Eng Comput 25:155, 1987). The Frank vector diagram can be predicted for all people or patients using a single set of equations.

When creating the regression equations from a data set, overfitting must naturally be avoided. Non-linear methods such as the “support vector regression method” (KKU Engineering Journal, 2016; 43 (S2): 317-322) or similar non-linear methods can also be realized and often show smaller deviations of the results. Here too, neural networks have proven themselves well, because they are better in representing non-linear relationships.

In order to take differences in the shape of the torso into account, a three-dimensional recording of the torso, for example with a 3-D camera, can be very helpful in order to be able to record deviations in the shape of the torso from a cylinder and the exact arrangement of the electrodes in three-dimensional space.

It is advantageous if the support surface comprises a plurality of at least partially convex and/or concave, preferably elastic, deformations, which carry electrodes. This enables particularly good electrical contact between body parts of a person to be examined and the electrodes.

It is advantageous if at least one electrode is attached to a deformable material, which deforms when a person is positioned in order to establish a favorable electrical connection of the electrode to the human body. Appropriately designed electrodes can basically be provided at any position, especially in the area of the wrists or forearms and just above the ankles or on the lower leg just below the knee joint. The electrodes can also be attached in semi-cylindrical shells lined with deformable material, which cylinder axes correspond to Have longitudinal axes of the associated extremities so that a person to be examined can easily position the arms and legs in the shells, while at the same time a favorable electrical connection is established via the deformable areas. In order to avoid any kind of mechanics despite the possibility of choosing the optimal electrode position, several U-shaped, concave support surfaces could be arranged on the arms and legs, which can optionally be selected using a multiplexer. These electrodes for the relevant extremity would then not lie on the respective electrode axis, but depending on the distance to the base of the extremity, outside the extremity axis, so that they do not interfere with each other. The examined person then selects, for example, the U-shaped electrode-bearing support surface just above the wrist or above the ankle by alternating addition and abduction of the arms and legs. Depending on which electrodes form a closed circuit or which have the lowest contact resistance, or at If any built-in pressure sensors at the electrode position signal the greatest contact pressure, the corresponding electrodes are activated and the others are switched off. When a person lies down on the support surface, the electrode pairs are forcibly created, in which the arms and legs fall best. This means that a continuously closed support surface can be created between the support surface and the support surface without any interruptions, corresponding to a relief shaped after the body, which is absolutely necessary for hygienic reasons, since, for example, the support surface could be cleaned, for example also sprayed down. In addition, all electronic components and cables can then be welded between the support and support surfaces, which further increases the safety of the device. The support surface can best be stitched transversely in the center, so that it can be folded and transported well.

As an elastic material, any suitable material can generally be used. It is particularly preferred that at least one electrode is arranged on a skin-friendly, robust elastomer. In order to achieve particularly good contact, it can also be provided that at least one electrode is arranged on a convex area that protrudes from the support surface. Therefore, good contact can be achieved, particularly in the area of the neck, for example in the form of an electrode placed on an elastic, possibly movable neck roll, or in the area of the upper body, especially since a correspondingly protruding, convex area results in an increased pressure load on this area, which is for ensures good contact. The contact caused by body weight is particularly critical, which is why particularly corrosion-resistant and very well conductive electrodes with low contact resistance, such as gold-coated electrodes, should be used. In particular, this makes it possible in a simple manner to carry out a corresponding diagnosis even through items of clothing (particularly if they are conductive or moistened). This is particularly ensured when the contact region is moistened, for example by electrode spray. Therefore, the diagnostic system can be used in particular in public areas such as a shopping center, waiting areas, etc.

It is preferably provided that at least one electrode, in particular the electrodes on the leg areas and the arm areas, are concave in order to provide a corresponding area of the human body, in particular a leg area and an arm area. Through a corresponding, for example U-shaped design, which can be formed in particular by a part of a hollow cylinder, good contact can be achieved on the one hand and at the same time favorable handling on the other. Hands and feet can also be used, for example with electrically conductive handles, as electrode positions, especially for the feed, and also as positions for the measuring electrodes, which, however, means an increased series resistance.

In order to achieve particularly good contact, it is preferably provided that the electrodes are easily deformable, e.g. made of conductive rubber or fabric.

The diagnostic system can be easily adapted to people of different sizes if at least two electrodes are movable relative to one another, in particular the electrodes on the leg areas relative to the electrodes in the upper body area and/or the electrodes on the arm areas relative to the electrodes on the upper body area, in order to adapt the diagnostic system to people of different sizes. This adaptability can be achieved, for example, by parts of the support surface that are connected in an articulated or displaceable manner to a main body or by separate support surfaces on which electrodes of the leg areas and/or electrodes of the arm areas are arranged.

Adaptability to people of different sizes can also be achieved in a simple manner as an alternative to a mechanism with which individual electrodes can be moved relative to other electrodes if several adjacent electrodes are arranged on the leg areas, the arm areas, the neck area and/or the upper body area, wherein the device is set up to activate one or more of these electrodes depending on the size of the person to be examined, with the electrode being activated for a measurement which has the best electrical contact with the respective body area and/or which is anatomically correctly located. For this purpose, for example, a pressure sensor, in particular piezo sensors, can be additionally provided on the electrodes in order to determine those electrodes on which a mechanical pressure caused by the body weight of the person on the diagnostic system is greatest At a corresponding position, an optimal electrical contact or an electrical contact with the lowest contact resistance is usually also achieved. It is preferably provided that at least three electrodes are provided in the upper body area at approximately the same height, wherein at least one electrode is preferably being arranged approximately in the center and the two further electrodes being arranged approximately symmetrically to the side.

Impedances in the area of the upper body can thus also be accurately detected. In addition, this arrangement can also easily generate an ECG with the diagnostic system.

In order to be able to determine the state of health of a person to be examined particularly precisely, it can also be provided that an upper arm cuff and/or a pulse oximeter are provided. Such devices can be designed simply, for example with a Velcro fastener or an elastic clamping device, so that a person to be examined can also put them on themselves. The system can therefore still be operated without personnel.

The pulse oximeter can, for example, be designed to detect oxygen saturation on a finger or another part of the body, for example on the wrist, on the forehead, etc. and, if necessary, be integrated into VR glasses. In order for these glasses to be able to be used by different people within short intervals, sterilization is necessary; this can be done within 60 seconds within a “clean box”, in which the glasses are sterilized and dried for the next person, e.g. by means of UV radiation and drying. After release, for example through payment, the person using the device receives a code or electronic key with which the clean box is opened, the 3-D glasses are released and the examination is activated.

It is advantageous if the support surface is made of a washable plastic, preferably a deformable material, and if there is a support surface below the positioning surface, which is made in particular of durable fabric.

The support surface can also be designed to be pressurized with a changeable pressure, for example by means of a chamber between positioning and support surface, which is filled with fluid, e.g. gas, by means of a compressor. Particularly in the area of the extremities, a U-shaped shape of the support surface is sufficient to exert pressure on the artery from the outside, for example hydraulically, in order to measure the oscillations of the pressure with the corresponding counterpressure and, for example, to determine the blood pressure oscillometrically and/or auscultationally or to perform a pulse wave analysis. This is particularly possible on the wrist, for example, since there is a osseous boundary of the arm on the open side of the U. To prevent the arm from being pressed out of the U, an area in which no hydraulic pressure is exerted could alternatively be provided on the base of the U.

It is advantageous if at least one, preferably each, electrode is arranged on an area which can be pressed against a human body with variable pressure, the area being designed in particular as a bladder that can be filled with a fluid, with a pressure of the fluid in the bladder can be changed, preferably by means of a pump. As a result, a good contact can be achieved between the electrodes and a human body. The arrangement of the electrodes on corresponding areas, in particular on inflatable chambers or bladders, can only be done with individual electrodes, for example on the electrodes in arm areas, leg areas, chest areas or even on all electrodes. In particular, it can be provided that the electrode, which is arranged in a neck support or a neck roll, can be pressed against a person over such an area or a bladder with variable pressure in order to enable precise measurement in the area of the neck roll. Accordingly, electrodes in the region of a chest strap with variable pressure can also be pressed against a person. In particular, a chest strap can be designed with at least one inflatable chamber for this purpose.

For this purpose, one or more pressure sensors can be provided, which can detect pressure changes in the fluid. Furthermore, a device for changing a pressure in the fluid can be provided, in particular a pneumatic and/or hydraulic device, with a pressure sensor also being provided for determining a pressure in the fluid.

It is advantageous if the diagnostic system has sensors with which the mechanical behavior of body parts of a person located at the diagnostic system on applied forces and/or movements can be detected in order to be able to assess mechanical stiffness of individual body areas, in particular depending on a frequency with which a force and/or a movement are applied. In this way, in addition to the impedance determination, with which electrical properties of the individual body regions are determined, additional information can be provided with regard to a mechanical composition of the individual body areas, for example to be able to determine a fat content or a muscle mass or a bone density. In addition, it has been shown that, for example, liver disease can be inferred based on the rigidity of the liver, so that statements can be made about the health status of the person or part of the person's body based on the mechanical parameters that can be determined. Forces and movements can be applied to the individual body areas, for example with vibrating devices, which can optionally vibrate at different frequencies and/or different amplitudes or force intensities, or other devices, which are known, for example, from massage devices.

If the reaction of the examined person to the applied physical forces, such as heat, cold, electric current, pressure, vibration, is recorded, for example by acoustic, optical or tactile communication, the function of the nervous system, for example the sensor system or perception, can also be recorded of the person examined can be recorded and quantified.

It has proven to be particularly comfortable if the diagnostic system is designed as a bed. in particular as a tilting bed, with a tilting angle being detectable. On the one hand, this is a comfortable way for a person to be examined. On the other hand, the tilt angle can also be used to easily determine a hydrostatic pressure difference between individual areas of the body, so that measured data can be interpreted even better or can be corrected for a hydrostatic pressure difference. This can also be used to determine whether there is an adequate reaction of the circulatory system and the autonomic nervous system, e.g. stroke volume, heart rate, vascular resistance, sympathetic nervous system, parasympathetic nervous system, etc., to the tilting maneuver.

In order to be able to analyze a person to be examined beyond the impedances of the individual body regions, it is particularly advantageous if one or more cameras, in particular also an infrared camera, and/or time-of-flight sensors are provided, by means of which a person located on the support surface can be three-dimensionally determined with which dimensions of an examined person can be determined. A camera can also be used to record the skin color of the person being examined and to derive medical information from it. By detecting a three-dimensional contour of the person to be examined, the size of the person to be examined and the body segments can, for example, be recorded particularly precisely, which in turn enables those electrodes for impedance determination which are arranged according to the respective size to be activated. With the help of the size and length of the body sections measured in this way, the position of the electrodes, especially the neck electrodes on the neck roll and on the legs, could also be automatically brought into the correct position using auxiliary motors, for example. This also provides bidirectional communication between the imaging method and the electrical and other sensors, which further improve the measurement results and diagnoses. In addition, the three-dimensional contour can be used to determine additional information for determining the circumference and diameter of the body segments, body fat, a body mass index or a waist-to-hip ratio, which values are also meaningful for a health status.

In order to make the data obtained with the diagnostic system according to the invention even easier to evaluate, an additional 3-D measurement of the human body can be of great advantage. Recently, methods for non-contact measurement of the body using optical methods have improved greatly in this regard. The human body can be represented using normal photographs, preferably in several planes, stereo cameras, or time-of-flight methods and, when using a scale, the body dimensions can be represented in 2-D or 3-D. Once the body dimensions can be included in the evaluation using contact sensors, these two methods can be combined.

For example, the electrical resistance of the body or one of its segments is not only dependent on the specific resistance, but also on the length and the cross-section of the examined body part or body. The combination of these two measurement methods thus provides even more precise biological data. The two combined methods also enable feedback between the results of the non-contact measurement and the contact measurements.

For example, the localization of the electrodes could be improved through direct feedback from the contactless measurement and vice versa. It could also be possible, depending on the results of the non-contact measurements, to measure additional segments of the body, for example by activating additional electrodes at other locations, such as the proximal lower leg to detect a circulatory disorder or in regions that are particularly affected by the non-contact measurements appear critical. For this purpose, electrodes can be arranged distributed over an entire support surface of the diagnostic system, i.e. over a surface on which a person positioned as intended on the diagnostic system touches the diagnostic system, which can be switched individually in order to determine impedances of individual body regions. The electrodes can usually be switched close to the surface or close to the coupling point.

In particular, it is useful to take a thermographic image of the person, for example, to detect a circulatory disorder, especially in the lower extremities, local inflammation caused by thrombosis, or an infection, which cause warming. Furthermore, traumatic or inflammatory changes in the joints or local tumors can be detected by means of a warming. Not only the optical method, but also the thermographic method is particularly suitable for this. A thermal imaging camera could also be used to improve the three-dimensional representation of clothed persons.

Therefore, a thermal imaging camera is also preferred, with which the temperature of a person on the diagnostic system can be detected. On the one hand, this can be used to determine whether the person has an elevated temperature or fever. In addition, the recording of temperatures on a surface of the person to be examined can also be used to draw conclusions about inflammations, tumors and/or circulatory disorders. The temperatures can be measured with a thermal imaging camera and/or temperature sensors, which can be integrated into the support surface, for example.

A chest strap is preferred, which has external chest strap electrodes on the outside that correspond to contact electrodes on the support surface, so that electrical signals from the chest strap can be transmitted via the external chest strap electrodes to the contact electrodes on the support surface.

In a conventional ECG, electrodes are placed on the front of the person to be examined on the upper body. The inventor has determined that it is basically possible to create an electrocardiogram with electrodes arranged on the back, which in this case can be positioned in an upper body region. This is another way in which a high level of accuracy can be achieved. For special cases, however, it may be advantageous to also use a chest strap that can be used to take electrical measurements on the front of the person being examined. The chest strap is then preferably designed in such a way that it can be put on by the person to be examined, so that the device can continue to be operated without personnel. Accordingly, contact between the chest strap and the electrodes of the support surface is also simple and, in particular, does not require an additional plug contact, but only a loose contact, so that the diagnostic system can be easily operated and administered by the person to be examined.

It is advantageous if an optical medium, e.g. a screen or a VR headset, is provided in the field of vision of a person positioned at the diagnostic system, which is connected to sensors of the diagnostic system via a data link, so that data relating to the person's state of health, in particular, can be displayed on the screen. The diagnostic system thus preferably has a data processing device that is connected to all electrodes and sensors as well as the screen. Based on the data obtained with the sensors and electrodes, this device can provide information about the state of health of the person being examined, which is usually displayed in real time. A combined display of the optical sensors of the 3D representation with the representation of the data as obtained from the measured values is particularly advantageous.

The following diagnoses can be made, for example: all diagnoses made using the 12-channel ECG, e.g. stroke volume, cardiac output, vascular resistance, heart attack, coronary heart disease, pulmonary embolism, pulmonary congestion, pneumonia, myocarditis, pericarditis, right heart overload, heart valve defects, Lown classification, arrhythmias, atrial fibrillation, risk of atrial fibrillation, etc. Furthermore, blood pressure (e.g. via an integrated blood pressure monitor or via a U-shaped support surface to be applied with variable pressure in the region of arms and legs), central blood pressure via a transfer function of the peripheral blood pressure curve or via a modified wind chamber model (McEniery European Heart Journal (2014) 35, 1719-1725). Cardiac output and the detection of heart failure can be recognized by form analysis of the plethysmograms with lower amplitudes and slopes (Skrabal F et al, Med Eng Phys. 2014 July; 36 (7): 896-904). For this purpose, it is planned to use mean value curves, so-called templates, from the raw data, and to use artificial intelligence, e.g. deep convolutional neural nets, genetic algorithms in addition to conventional mathematical methods such as discriminant analysis, etc., in order to improve the accuracy of all calculated parameters.

Heart failure can also be recognized by changes in breathing with prolonged expiration, dry and moist respiratory sounds, by changes in extracellular water in the segments (Skrabal F et al Med Eng Phys. 2014 July; 36 (7): 896-904), by the discovery of low muscle mass, etc. Lung function with the commonly known parameters FVC, FEV1, PEF, MEF25, MEF50, MEF75 and MMEF can be measured via the breathing tube, breathing gases via CO2 and O2 sensors. The characteristic curve of the CO2 exhalation curve can be used to detect lung diseases, especially COPD (Mieloszyk, IEEE Transactions on Biomedical Engineering 61, 2882-2890. 2014). Oxygen saturation can be measured via an O2 sensor, specifically at the electrode positions on the extremities or on the ear, preferably integrated into the band of the VR goggles. Vision testing is done by changing the size of objects in the visual medium and observing whether the patient recognizes them or not, specifically using VR goggles with feedback (acoustic feedback in the form of speech or by having the patient press a button). Hearing testing is done by changing the volume in the acoustic medium, detection or non-detection and corresponding feedback), heart rate variability (via spectral analysis of heart rate intervals), polyneuropathy by measuring the sense of touch (altered impulses through actuators at the support surfaces with feedback by the examined person), sense of temperature (altered temperature at the support surfaces with feedback), sense of vibration (altered vibration at the support surfaces with feedback), autonomic nervous system for controlling the circulation with sympathetic and vagus nerves via a spectral analysis of heart intervals and blood pressure fluctuations with special evaluation of the 0.1 and 0.3 Hz bands, baroreceptor reflex sensitivity via spontaneous blood pressure steps using heart rate intervals, statements about hydration status, muscle mass, muscle strength, biological muscle age, fat mass, abdominal (visceral) fat (Skrabal F et al, Med Eng Phys. 2017 June; 44:44-52) to assess metabolic and diabetes risk (characterized by high waist-to-hip ratio, low muscle mass, high fat mass, high abdominal fat), thrombosis, lymphedema (characterized by changes and asymmetries of extracellular water in the legs). It is possible to measure the pulse wave transit time over the time of propagation of the pulse wave or volume wave (Skrabal F et al, J Hypertens. 2020 October; 38 (10): 1989-1999), as well as analyses of the shape of the pulse wave and a wind-cavity model (Cohn J, Hypertension. 1995 September; 26 (3): 503-8) or central aortic pressure (O'Rouke, Am J Hypertens. 2014 February; 27 (2): 143-5), or the method of flow-mediated dilation (FMD) to assess vascular function via the applied hydraulic compression of the arteries and veins (FMD and venous occlusion plethysmography), continuous beat-to-beat blood pressure via vascular unloading with measurement of arterial blood flow using a pulse oximeter and volume clamp also using the U-shaped support surface (Penaz J, Voigt A, Teichmann W, Z Gesamte Inn Med (1976) 31:1030-3), ankle-brachial index (by measuring blood pressure in the arms and legs), circulatory disorders in the legs (using different plethysmograms of the legs), central aortic pressure, e.g. via a transfer function of the peripheral blood pressure or a modified wind-cavity model, furthermore, carotid artery disease via flow murmurs in the U-shaped support surface on the neck, atherosclerosis, e.g. via pulse wave transit time, pulse wave analysis, ankle-brachial index, liver compaction using a Fibroscan (elastography), in which a low-frequency vibration of approx. 50 Hz is applied to the liver and the deformation of the liver is measured using a 5 MHz transducer (Piscaglia, Digestive and Liver Disease 49, 802-808, 2017). This requires the application of vibration to the back in the area of the liver, which should also be applied to other parts of the support surface anyway, as well as good contact with an ultrasound probe located there, preferably with ultrasound gel, etc. It is also much easier to assess kidney function if the lean body mass and the appendicular muscle mass are known, because serum creatinine values, as they can be measured by point of care, can be corrected for the different lean body mass. With low muscle mass and low lean body mass, the creatinine clearance is Incorrectly calculated as being too high using the MDRD formula and other formulas.

However, with knowledge of muscle mass, an unadulterated determination of creatinine clearance is possible. It is also possible to achieve a better drug dosage, e.g. for cytostatics or narcotics, because many drugs are dosed according to lean body mass or fat mass. It can also be used to calculate the biological muscle age, for which there are standard values. An unexplained loss of muscle and/or fat during follow-up with the diagnostic device; localized heating may also indicate consuming diseases such as autoimmune diseases, inflammations, infections, occult tumors, malignancies. Training monitoring with the progression of muscle mass and the calculated aerobic capacity (calculated from age, waist-hip ratio, e.g. measured with a 3D recording of the examined person, height and shape of the plethysmogram, measured muscle mass, fat mass, abdominal fat, etc. using multiple regression equations or artificial intelligence) is very good and is already being used by us (see figures). Many other diagnoses, not mentioned here, can be made. Even foci of inflammation in the body, e.g. using thermographic methods, or diabetic foot syndrome, can be detected using thermography and pulse amplitudes, brachial index.

In the context of point-of-care measurements, metabolic diseases such as diabetes, bone metabolism disorders and disorders of all internal organs are also possible. Malignancies can also be easily detected, for example, by the fact that unexplained changes in fat or muscle mass can suddenly be observed during follow-up examinations, which can be carried out automatically thanks to the permanent storage of data. These are just a few examples of the many applications that are possible with this diagnostic system. Other possible applications are by no means limited to these examples and are known to experts.

Virtual reality glasses, which may themselves be equipped with biosensors and are connected to sensors of the diagnostic system via a data link, can also be provided for displaying information and for a person's interaction with the diagnostic system, so that data relating to the person's state of health, in particular, can be displayed using the virtual reality glasses.

In this context, it is advantageous if the virtual reality glasses have sensors for recording health-related data of the person, in particular sensors for recording physical quantities such as electricity, light, gases, flow velocity and the like. Thus, virtual reality glasses can also contribute to data collection and diagnosis, so that an effect beyond the mere presentation of information is achieved.

Preferably, the virtual reality goggles are equipped with a breathing tube that includes the nose and is used to measure flow rate, breathing pressure, breathing volume and a gas analysis. Thus, for example, lung function can be easily determined with the diagnostic system in order to obtain as complete a picture as possible of a person's state of health. Differential pressure, turbine flow measurement and ultrasonic flow measurement are common methods for this, wherein any future development in this field can also be integrated here. The ultrasonic flow measurement is usually performed in sensors placed approximately along the respiratory flow by measuring in both directions, taking into account the molar mass of the gas and the temperature.

A replaceable mouthpiece for measuring lung function and respiratory gases could also be used in the usual way, although this would make the diagnostic system less elegant. Also brain function disorders such as epilepsy via the glasses and their band-accommodated EEG electrodes, brain function deficits, visual testing, hearing testing, etc. are possible via the use of virtual reality glasses and bidirectional communication, e.g. also via speech, if these are equipped with additional sensors.

For efficient diagnosis in complex cases, a bidirectional communicating online expert system for the diagnosis of numerous illnesses may be available, to which the diagnosis system is connected via a data connection, in particular via the internet. This means that experts or an automated expert system can be consulted if necessary, to whom all measurement results determined by the diagnostic system can be made available, for example via the internet. For example, a cell phone can be used to activate a person's measurement on the one hand, and on the other hand, the results can then also be communicated.

Mathematical methods for determining impedances from currents applied to the individual electrodes and voltages measured are sufficiently well known from the state of the art.

Preferably, it is envisaged that electrical lines to the individual electrodes and/or sensors, in particular pressure sensors, light sensors, sound sensors, actuators and the like, are arranged between the bearing surface and the support surface. This bearing surface with the support surface could also be designed as a mat, for example, which could possibly also be reduced in size by rolling or folding so that it can be easily transported and placed on any surface. A directional bearing surface with a crease in the longitudinal center of the mat (in the hip region of the body to be placed) would be enough to create an easy-to-transport package. Preferably, all electronic components, sensors, cables, etc. are welded or glued between the support surface and the bearing surface. In order to securely attach the camera or measuring device to the mat above the examined living being, stable brackets can be attached to the mat, e.g. a pipe, to which the bracket for the camera or imaging device is removably attached.

To achieve a particularly pleasant surface, it is preferably provided that a fluid is arranged between the bearing surface and the support surface. This results in particularly good pressure distribution. This can be achieved, for example, by inserting a gas, in particular air, or a liquid, in particular water, between the bearing surface and the support surface. To achieve a favorable contact, it is preferred that the electrodes are designed as strip electrodes. Such electrodes have a length-to-width ratio of at least five, preferably at least ten, to achieve favorable electrical contact.

It is advantageous if microphones, pressure sensors and/or acceleration sensors are provided at the support surface, in particular to be able to monitor breathing, for example, via the breathing sounds of a person located at the diagnostic system. Furthermore, pressure sensors can also be used to obtain information about the weight, size and dimensions of the individual limbs of a person to be examined, which can be used to make a diagnosis.

The diagnostic system can also be used to determine the state of health of the nervous system of the person being examined. For this purpose, it is preferred that devices be provided to generate predefined temperatures and pressures or vibration signals at different positions, in order to be able to determine reactions of body parts of a person located on the diagnostic system to applied temperature changes, mechanical effects via feedback from the person being examined. For example, the diagnostic system can be designed to apply a (slightly) lower or higher temperature than body temperature or mechanical signals of varying intensity via these devices, while at the same time obtaining feedback from the person being examined as to when and where they perceive this temperature change or mechanical influences, e.g. through a sense of touch, depth sensitivity or vibration. This is a simple way of measuring the conduction velocity and the health of nerve pathways.

For this purpose, it is preferable to provide devices for emitting acoustic and/or optical signals to enable communication with a person on the diagnostic system. This can be achieved, for example, using loudspeakers and/or a screen, as well as optional VR goggles. Accordingly, microphones and input devices are usually provided so that the person being examined can also provide feedback to the diagnostic system.

It is particularly preferred if the device is configured to reproduce a three-dimensional image of a person at the diagnostic system that has been captured by a camera. For this purpose, a screen and/or VR glasses can be used, with which images and videos of the beating heart, the pulsating aorta and the breathing lungs of the examined person can be observed in the transparent and realistically displayed person.

Typically, the diagnostic system is configured to display a diagnosis in real time, for example by coloring a region of the body on a screen in the field of vision of the person who is on the diagnostic system, according to the state of health of the respective body region of the person.

It is advantageous if the electrodes are designed for contactless recording of measured values for an ECG, in particular as capacitive electrodes. This makes it possible to collect health-related data even through clothing, which is particularly advantageous for the application of the diagnostic system according to the invention in a public place such as a shopping center.

Preferably, the diagnostic system is designed to use the electrical measurement values determined with the electrodes and/or contactlessly determined measurement values, in particular measurement values determined with a camera, to determine the lean body mass and/or muscle mass and to correct a kidney function based on the lean body mass and/or muscle mass. This means that data on kidney function determined using conventional measurement methods can be easily and quickly corrected for any higher or lower muscle mass, which is not taken into account in a conventional assessment of kidney function.

It has been shown that one, preferably two, microphones are provided in a chest region, which can be pressed against an area of the human body, preferably by means of areas with variable pressure, in particular bladders filled with a fluid, in order to detect lung sounds. This allows lung function or pathological lung conditions, such as pulmonary congestion, pneumonia or emphysema, to be easily and automatically determined.

Further features, advantages and effects of the invention will become apparent from the following exemplary embodiment. The drawings to which reference is made show:

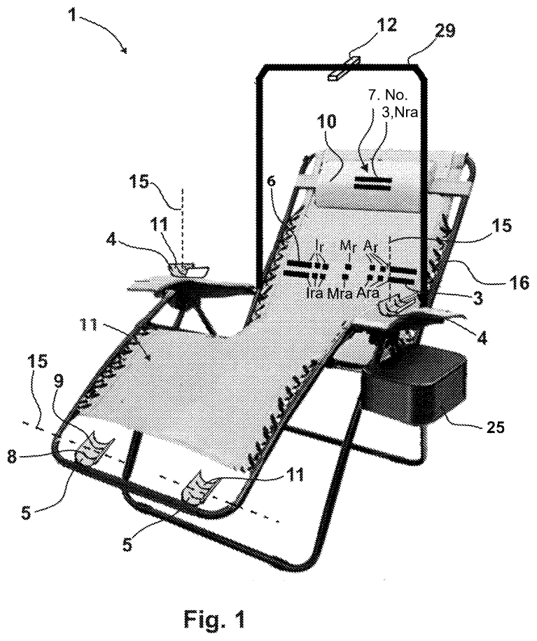

FIG. 1 an inventive diagnostic system;

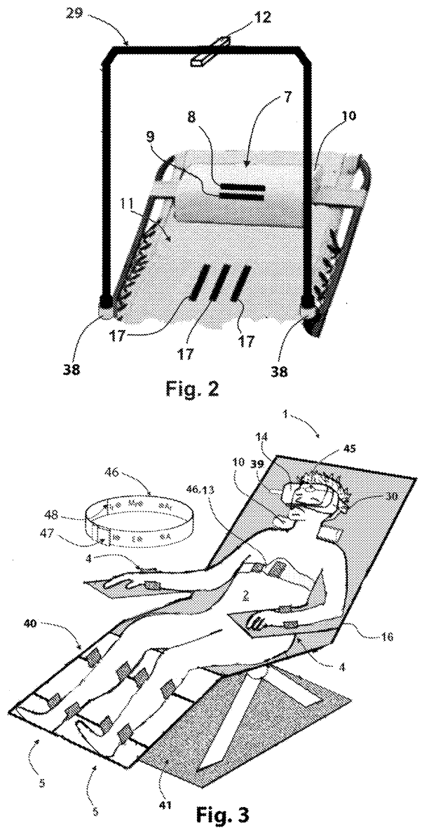

FIG. 2 a detail of the diagnostic system according to the invention;

FIG. 3: a diagnostic system according to the invention, together with a person to be examined;

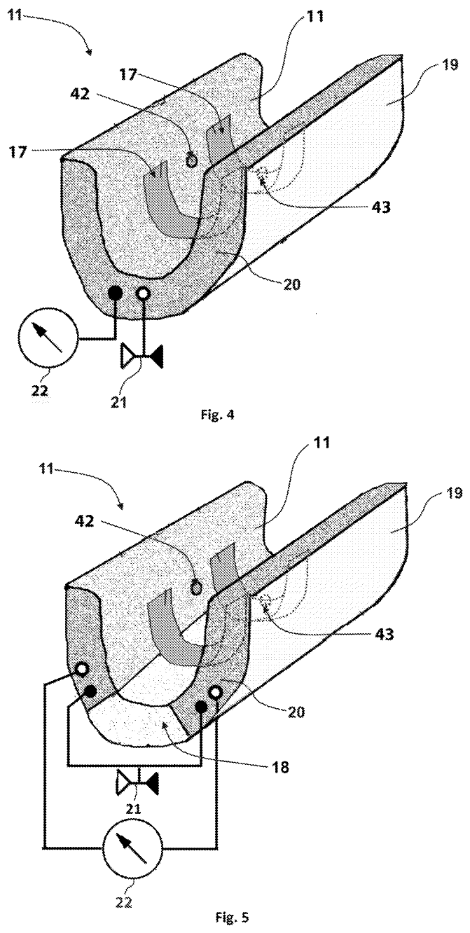

FIG. 4 a detail of the diagnostic system: undivided support surface for arms and legs FIG. 5: a detail of the diagnostic system: divided support surface for arms and legs

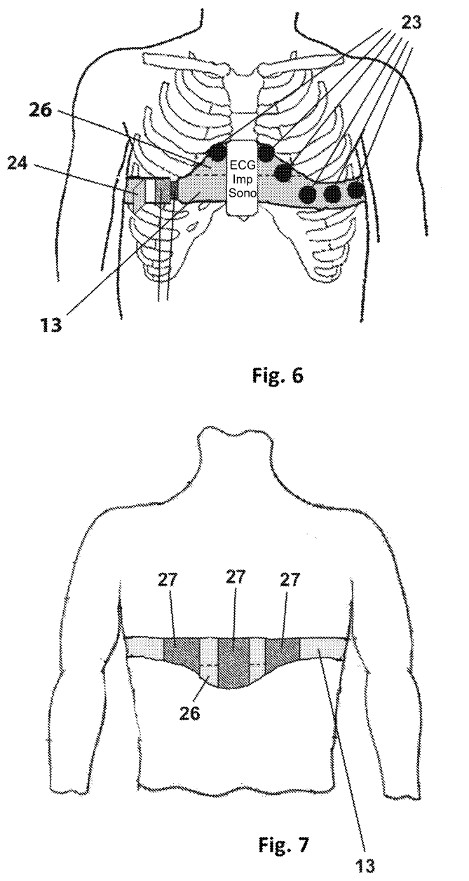

FIGS. 6 and 7: different views of a chest strap for the diagnostic system;

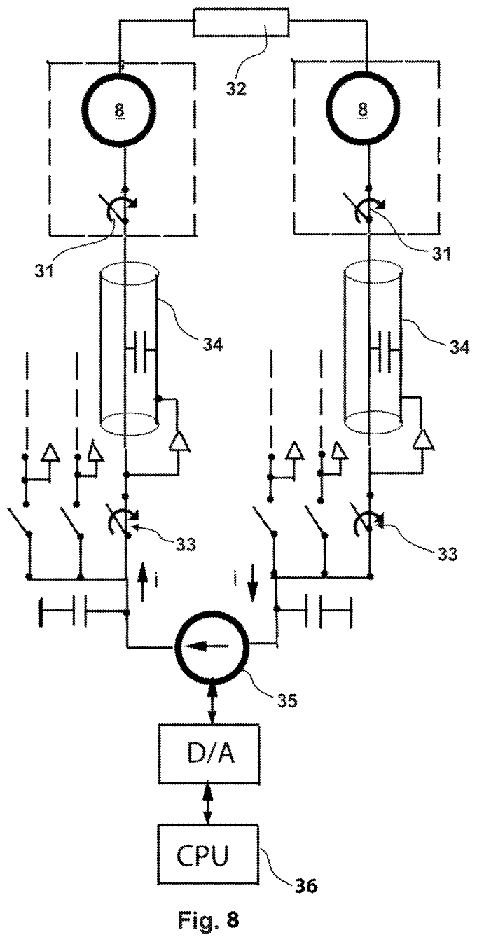

FIG. 8 a schematic circuit diagram of the diagnostic system with an AC power source;

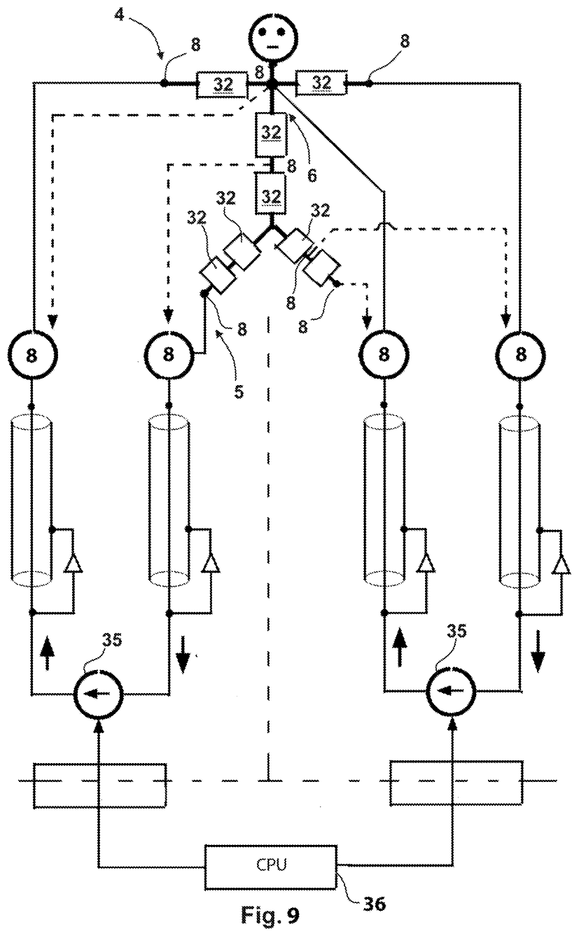

FIG. 9 a schematic circuit diagram of the diagnostic system with several AC sources;

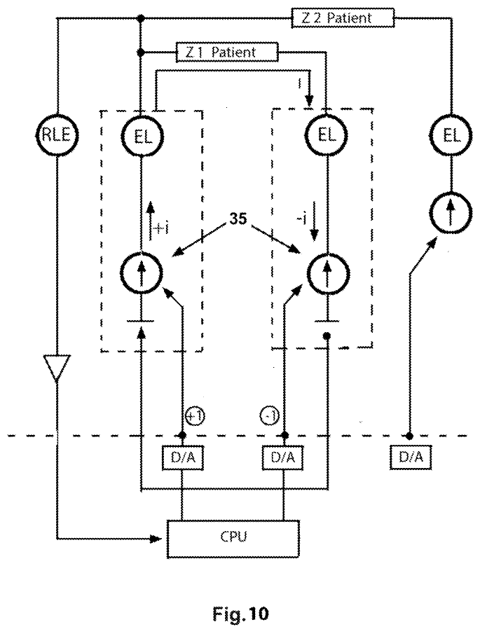

FIG. 10 a schematic circuit diagram of matched alternating current sources;

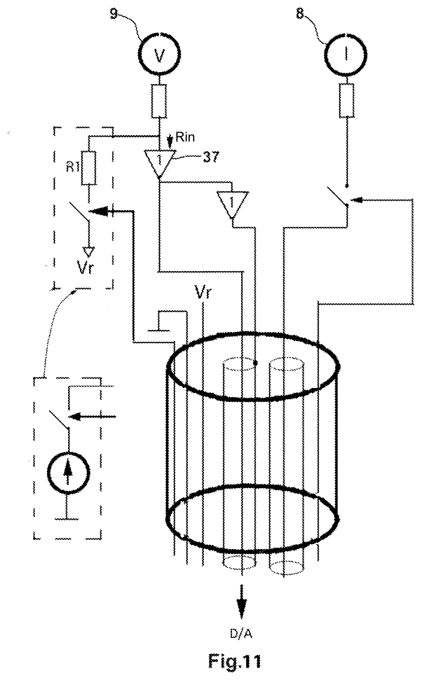

FIG. 11 a schematic diagram of a possible electrode contact measurement;

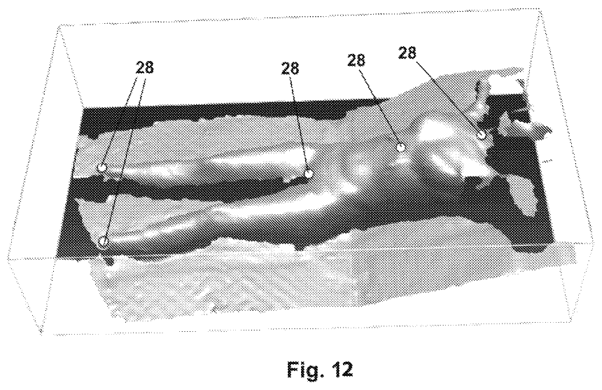

FIG. 12 a three-dimensional image of a person to be examined, recorded with the

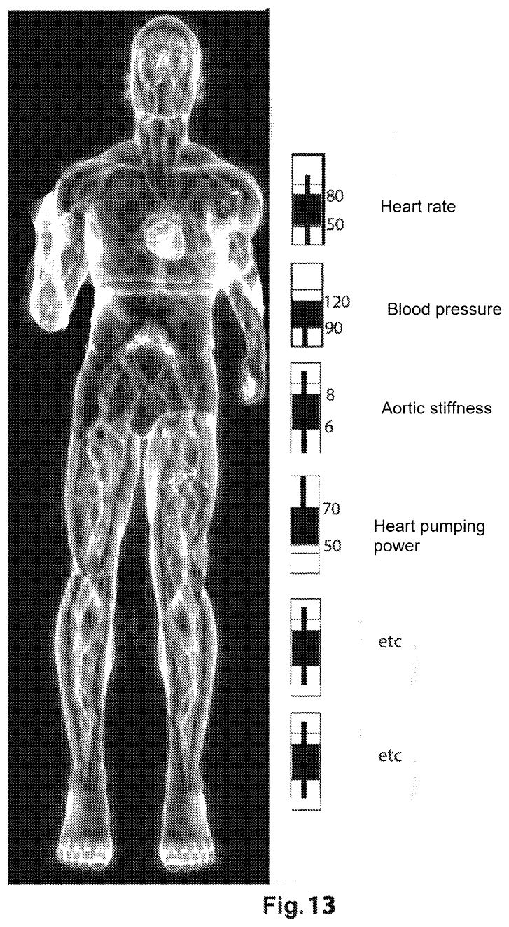

FIG. 13 a visualization created with the diagnostic system according to the invention;

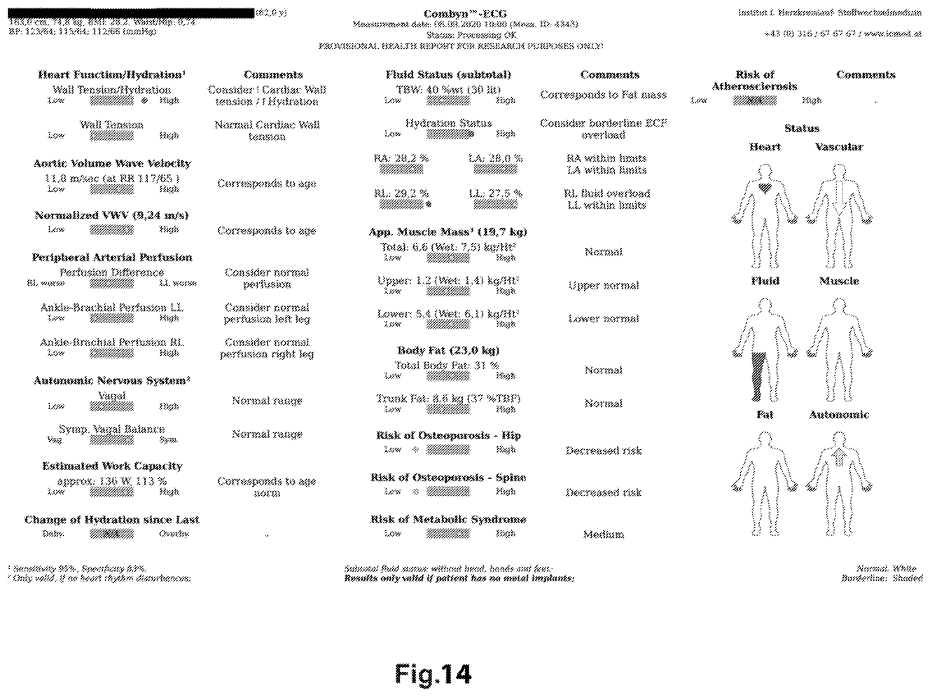

FIG. 14 through 17: Diagnostic reports created using the diagnostic system according to the invention.

FIG. 1 shows the diagnostic system 1 according to the invention, designed as a tilting couch with a tilting frame 16. As can be seen, the diagnostic system 1 has electrodes 3 arranged in leg regions 5, arm regions 4, an upper body region 6 and a neck region 7. This makes it easy to measure a resistance network or an impedance network of a human body.

In each case, one electrode further away from the center of the body is designed as a feed electrode 8 and one electrode closer to the center of the body is designed as a measurement electrode 9, so that an electric current, usually an alternating current, can be applied via two feed electrodes 8 and a voltage drop can be measured between the measurement electrodes 9 arranged in between.

The diagnostic system 1 is designed here, for example, as a tilting table so that a height difference between the upper body region 6 and the leg regions 5 can be easily changed, in particular by shifting a center of gravity. Furthermore, an angle sensor is planned that is not shown, with which this height difference can also be detected in order to determine a pressure difference caused by the height difference.

If the diagnostic system 1 is attached to a scale 41, the person's weight could be determined automatically at the same time. As is usual for couches, the diagnostic system 1 has two preferred bends, the lower bend intended for the legs is convex and the upper bend intended for the hips is concave. If a mechanical joint, e.g. a hinge joint, is provided at these points, it can be set in motion using actuators, for example, enabling physiotherapy of the joints with movement therapy. Such mechanical joints can be provided for all joints of the human body. The diagnostic system 1 can also be used to integrate a massage or vibration device, such as the “power plate”, into the examination couch in order to utilize the examination time for additional purposes.

The diagnostic system 1 has one or more support surfaces 11, on which a person 2 located on the diagnostic system 1 touches the diagnostic system 1 and in which support surfaces the electrodes 3 are arranged in the arm regions 4, the leg regions 5, the upper body region 6 and the neck region 7 and, in addition, possibly in the knee region 40. In addition to the electrodes, pressure sensors and devices for applying forces and/or movements, as well as sensors for detecting movements or, in particular, acceleration sensors, can be provided in order to detect not only an electrical condition of individual body regions but also a mechanical condition and thus to determine not only an electrical resistance network but also a mechanical assessment of the individual body regions. When the support surface 11 is designed as a mat that can be placed on any surface, it is desirable to design it as a single support surface, wherein this support surface then also includes arms and legs.

In the upper body region 6, electrodes labeled Iᵣ, Aᵣ, Mᵣ, Nᵣ and at a slightly varied height labeled Iᵣa, Aᵣa, Mᵣa, Nᵣa are shown, labeled accordingly, wherein these electrodes, because they are arranged on the back, are labeled with the additional index “r” and serve for the mathematical reconstruction of all ECG leads.

The electrically conductive electrodes integrated into the support surface 11 on the upper body region 6 are preferably deformable, in particular arranged on elastomers or formed by conductive elastomers, which establish contact with the person when he lies down on the support surface 11 of the examination couch.

Furthermore, two electrodes are shown, which are preferably placed in a support surface 11, which is designed here as a bolster 10, and which run preferably horizontally and parallel to the transverse diameter of the couch. These electrodes are used to inject alternating current to determine the impedance of individual body regions at different frequencies and also as ECG electrodes. The electrodes, for example, are made of electrically conductive material, electrically conductive rubber or electrically conductive plastic. The armrests, which form part of the support surface, are preferably fitted with concave support surfaces 11 for holding arm electrodes in arm regions 4.

The arm electrodes in arm regions 4 should preferably be placed just above the wrist, and the leg electrodes in leg regions 5 should preferably be placed just above the ankle. In order to achieve this with different arm and leg lengths, the concave holders that form support surfaces 11 for arms and legs are preferably designed to be movable, slidable and/or rotatable about one or more pivot axes 15, whereby the holders can adapt to the arm and leg length by the arm or leg being bent or stretched to different degrees.

For example, with long arms, the elbows would be bent outwards, while with short arms, the concave support surfaces 11 of the arms would be almost in line with the armrests. This may be necessary because the body dimensions of person 2 vary so much from person to person. These electrodes are used to apply impedance current and for impedance measurement, as well as for the Einthoven and Goldberger leads I of the electrocardiogram. Similarly concave support surfaces 11, which also represent part of the support surface 11, can also be found for the leg electrodes in leg regions 5 and knee regions 40, which are also attached to the examination couch. These concave support surfaces 11 are also preferably movable, preferably designed to tilt around a pivot axis 15, so that they can make optimal contact with the leg, regardless of its length. Alternatively, the examinee may be able to choose between several U-shaped support surfaces into which the arms and legs fit best, e.g., shorter arms can be examined in less abduction, i.e., left and right U-shaped support surfaces can be placed closer together, and a greater spread can be provided for longer arms and legs, thus placing the left and right support surfaces for the extremities further apart (or vice versa). This means that, depending on the arm and leg length, only the intended, e.g. U-shaped, support surfaces are available and the other support surfaces for the extremities are outside the extremity beam and do not interfere with the measurement. This design has the advantage that no moving parts are required and that a hygienically clean, completely enclosed support surface is available.

As schematically illustrated, the swivel axes 15 of the arm regions 4 are approximately vertical and the swivel axes 15 of the leg regions 5 are approximately horizontal.

For long legs, the support surface 11 of the legs would tilt away from the examination couch to accommodate the bent knees; for short legs, the support surfaces 11 of the holders would be approximately parallel to the examination couch or tilting couch.

The support surface 11 or the support surface 11 for arms and legs and the bolster 10 could, for example, also be equipped with additional sensors, for example for temperature, oxygen, or even pressure, or even actuators. A measuring module with a CPU 36 is preferred at the couch, which can not only contain a twelve-channel ECG, but also an alternating current source 35 with several frequencies, as well as a direct current source, e.g. for stimulation current therapy, which sends a measuring current to the activated electrodes, and at least one energy or pressure source and communication hardware.

In order to connect only the correct electrodes, i.e. those electrodes that are to be activated for an impedance measurement, to the current source in a measuring module with a CPU, a switch, preferably a multiplexer, can be provided to switch off the patient connection lines and the feed electrodes that are not required for the measurement in question.

This is advantageous because otherwise, especially at higher frequencies, parasitic effects on the circuit board and patient connection lines would create alternative current paths. This would subsequently result in measurement errors.

The measuring module with CPU 36 has circuits for multi-channel ECG, impedance injection and impedance measurement, multiplexer, evaluation unit for contact data and contactless data and is preferably placed as close as possible to the examination couch or examination mat.

Several galvanically isolated power sources can also be provided, which are connected to the individual electrodes, in particular to enable several measurements to be carried out simultaneously. The individual power sources can also be designed to apply currents of different frequencies to the electrodes, so that currents of different frequencies can flow through the individual electrodes if necessary.

The distance between paired electrodes 3 is preferably between 2 cm and 6 cm, ideally 3 cm to 4 cm. The electrodes in leg regions 5 and arm regions 4 can be used for the Einthoven and Goldberger leads and for impedance measurements and impedance injections. Furthermore, the electrodes can be used in particular in the leg regions 5 for the ECG leads II and II pursuant to Einthoven or Goldberger leads II and III and as reference electrodes. However, these leads can also be reconstructed from the so-called EASI leads according to Dower (J Electrocardiology Suppl. 1988, pp. 182-187) or can help to reconstruct the Wilson leads from the EASI leads. Our own experiments have shown that moving the EASI electrodes to the back of the torso and thus to the support surface 11 on which the examined body is resting also enables the conventional Einthoven, Goldberger and Wilson leads to be reconstructed. If the EASI leads are replaced by electrodes placed on the back, these can also be labeled as Iᵣ, Aᵣ, Mᵣ, Nᵣ leads (r for back or rear). It is favorable if the electrodes in the upper body region 6, -Iᵣ-, -Aᵣ, -Mᵣ-, are placed approximately at the level of the heart, which on the body surface corresponds to the lower part of the sternum, approximately the xiphoid. The electrodes -Iᵣa-, -Aᵣa- and -Mᵣa- can be adjusted to different body sizes by means of a height adjustment. In particular, regression equations, artificial intelligence or neural networks can be used to reconstruct the conventional leads according to Einthoven, Goldberger and Wilson, e.g. with regression equations.

The function of the electrode -S- according to EASI can be performed by the neck electrode Nᵣ (for the back of the neck), i.e. the electrodes 3 in the neck region 7 on the bolster 10, which is optimally located at the highest point of the bolster 10.

The electrodes 3, which are intended for the neck region 7, the arm region 4 and the leg region 5 and knee region 40, have two preferably parallel strip-shaped electrodes in the exemplary embodiment. The distance between paired electrodes 3 is preferably between 2 cm and 6 cm, ideally 3 cm to 4 cm. The electrodes in leg regions 5 and arm regions 4 can be used for the Einthoven and Goldberger leads and for impedance measurements and impedance injections. Furthermore, the electrodes can be used in particular in the leg regions 5 for the Einthoven or Goldberger leads II and III according to Einthoven or for the Goldberger leads, as impedance electrodes and as electrodes for the application of current.

For the reconstruction of the conventional ECG leads according to Einthoven, Goldberger and Wilson or the construction of so-called vector loops, it has been shown that the placement of the measurement electrodes -Iᵣ-, -Aᵣ-, -Mᵣ-, -Nᵣ- in the correct position is particularly important.

Therefore, it may be planned to use the optimally placed electrode from the electrodes -Iᵣ-, -Aᵣ-, -Mᵣ, -Nᵣ- and -Iᵣa-, -Aᵣa-, -Mᵣa-, -Nᵣa-respectively. The optical system described here can be used to optimally place the electrodes, or pressure sensors 44 near the electrodes can be used to indicate which electrode is ideally suited for the measurement. For the reconstruction of standard leads, it has proven useful to place the -Iᵣ, -Aᵣ, -Mᵣ, -Nᵣ electrodes in such a way that all three planes of space, namely the horizontal, vertical and sagittal axes (XYZ axes), are recorded three-dimensionally for the various leads. From these, all other leads can then be reconstructed using the xyz vector concept. The horizontal plane would be represented by the lead Iᵣ-Aᵣ, the vertical plane by the lead Mᵣ-Nᵣ and the sagittal lead by the plane Aᵣ-Nᵣ. To emphasize the three-dimensionality of the sagittal axis, the electrode Nᵣ could also be placed ventrally, for example in the area of the larynx, especially in combination with a mechanical or acoustic amplifier in the form of a microphone. To do this, the bolster, which is placed behind the neck, should also be U-shaped.

While it is possible in principle to place the Iᵣ, Aᵣ and Mᵣ electrodes lower on the support surface 11, this has the disadvantage that the electrode approaches the lower sections of the subject's torso, which would have to be uncovered as well. Therefore, the placement of the electrodes -Iᵣ-, -Aᵣ- and -Mᵣ- at the same height is advantageous. For a contact, it may be sufficient for a person to be examined to push up an item of outer clothing such as a shirt a little to ensure that the electrodes -Iᵣ-, -Mᵣ- and -Aᵣ- make contact with the body. Contact through damp or conductive clothing is also possible in principle.

A multiplexer could then enable the use of whichever electrode is optimally located. This part of the torso can be easily reconstructed using the 2D or 3D representation of the body, so that the optimal electrode can be selected after automatic evaluation of the representation of the body. The outermost electrode -Iᵣ-, -Iᵣa-, -Aᵣ-, -Aᵣa- or a marking for the same should preferably be extended outwards in the form of a band so that the outermost electrode is also visible for the 2D or 3D representation of the body in the 2D or 3D representation.

The electrodes located at the height of the trunk, Iᵣ, Mᵣ, Aᵣ, especially the electrodes located on the left side of the couch and trunk -Aᵣ- or -Aᵣa-, together with the double electrode at the neck region 7, can also be used to measure the impedance of the thorax and thus to measure the thorax plethysmogram. As can be seen from this document, some of the electrodes are active for current injection, and other active processes such as the application of heat or cold to check blood flow, vibration to test sensitivity or pressure to measure blood pressure and record the pulse curve may be provided. In particular, one of the support surfaces 11 for the arms, which are concave, could be used to change the pressure in this support surface 11 in a controlled manner using a hydraulic device or a stamp.

As shown in FIGS. 1 through 5, this support surface 11 would be designed to be deformable, for example in the form of an open, deformable U, not only to improve the contact between the electrode and the body, but also to be able to take a blood pressure measurement at the same time. For this purpose, this U-shaped support surface 11 would only have to have a relatively non-deformable bearing surface surface 19 on the outside, so that when the pressure increases, this pressure is preferentially exerted on the arm, especially the forearm, in order to measure blood pressure, in particular by oscillometry or auscultation. A continuous pulse curve can also be obtained in this way, for example by setting the pressure in the U-shaped support surface 19 close to the mean arterial blood pressure. A beat-to-beat blood pressure measurement can also be carried out using vascular unloading with the help of an optical sensor for blood flow. This U-shaped support surface 19 could also be used specifically for measuring temperature and oxygen saturation. For this purpose, the support surface 19 could preferably be designed to be approximately translucent, or the sensors could be placed in the inner wall of the support surface 19 facing the human body.

Furthermore, a conventional blood pressure measurement with an upper arm cuff can also be integrated into the device to measure blood pressure. Alternatively or in addition, a pulse oximeter or a multifrequency pulse oximeter for measuring all the parameters that are possible with it can be integrated into the diagnostic system 1. When the examined person 2 is placed on the couch, the necessary contact between the electrodes and the examined person 2 is established solely by the weight of the body parts. To accomplish this, it is advantageous that all electrodes used, for example ECG electrodes, impedance spectroscopy electrodes, impedance plethysmography electrodes, stimulation current therapy electrodes, e.g. also TENS therapy for pain treatment, etc., are placed on the support surface 11 or the support surface 11 of the arms in the arm regions 4 and the support surface 11 of the legs in the leg regions 5. For therapeutic purposes, such as mechanical effects on the body of the person, all areas of the support surface or the diagnostic system, such as, for example, a head hood, with or without virtual reality glasses, can also be used, in which the corresponding actuators are included. These could be physiotherapy devices that use a change in shape, particularly on the extremities and neck, to effect movement therapy on the joints as well, when the support surface itself has mechanical joints, particularly hinge joints, in the region of the joints and these mechanical joints can be moved by actuators. The diagnostic device can also be designed as a massage therapy device or a vibration therapy device if the appropriate devices are attached to the support surface. Or with the help of electricity, electrotherapy, e.g. electrical stimulation or pain therapy, can be carried out, for which the existing electrodes and contact electrodes can be used. For example, pulsed ultrasound therapy has also proven effective for treating neurodegenerative diseases such as Parkinson's or Alzheimer's, because it can apparently temporarily open the blood-cerebrospinal fluid barrier, thus enabling the removal of harmful metabolic products (Adv. Sei. 2020, 7, 1902583). For this purpose, a hood for the person being examined would be advantageous.

The first use of the diagnostic device could serve the diagnosis, these and further applications then a targeted therapy. The communication medium, e.g. a screen, VR glasses, loudspeakers or headphones, could display the biological and medical findings during the first use. In the first and subsequent applications, information could also be provided, particularly in the areas of relaxation, biofeedback, knowledge and advertising. In doing so, the information obtained from the diagnostic device could be given special consideration, e.g. stress reduction in the case of high sympathetic nervous system, nutritional advice for obesity, training and recommended training equipment for deficiencies in the muscular system, targeted person-specific advertising depending on the findings, especially when an anamnesis is included, etc.

By measuring the different body segments at different frequencies of alternating current, it is possible to determine very precisely body water, extracellular water, edema, muscle mass, muscle mass corrected for extracellular water, “dry muscle mass”, and fat (Skrabal F et al, Med Eng Phys. 2014 July; 36 (7): 896-904, Skrabal F et al, Med Eng Phys. 2017 June; 44:44-52). Muscle strength and biological muscle age can also be estimated from the data. A built-in or connected dynamometer can also be used for this. As is well known, only a few frequencies are needed to construct a Cole-Cole plot for an impedance spectroscopy. For 32-plethysmography, the pulse-synchronized volume changes in the individual body segments are recorded, among other things, as well as the change in volume before, during and after an externally applied pressure, so that not only cardiac output (Skrabal F et al, Med Eng Phys. 2014 July; v36 (7): 896-904), vascular properties such as pulse transit time (Skrabal F et al, J Hypertens. 2020 October; 38 (10): 1989-1999), but also arterial and venous circulatory disorders can be detected.

This support surface 11 could also be shaped as required to establish even better contact with the examined body, but it is essentially still shaped as a flat surface without any folds. This support surface 11 could be single-layer, as shown in FIG. 1, or multi-layer. All the necessary electrical connections can be made between the layers without any voltage, for example by making the cables longer and, if necessary, spiral.

In order to keep this support surface 11 hygienically clean, it may be necessary to make this support surface 11 as homogeneous as possible and without interruptions. This ensures that the next person to be examined can be cleaned quickly and safely.

The support surface 11 for the torso, arms and legs should be relatively non-deformable, so that a non-contact representation of the body is not distorted by excessive deformation of the support surface 11.

It is advantageous if a contactless 3D measurement of the patient can be carried out at the same time. A device for the contactless 3D measurement of the body or for further representations with the aid of a camera 12, possibly a 3D camera 12 or a thermal imaging camera 12 or time-of-flight measuring apparatus 12 for the representation and measurement of the person 2 being examined and for the representation of color and temperature, with which a person 2 positioned on the couch can be detected, could be attached to a bracket 29 mounted above the couch. Person 2 can be recorded in three dimensions, so that, in addition to impedances of the body regions of the person to be examined, further data on person 2 can be recorded, such as a volume. In addition, the color of the skin of person 2 can be determined using camera 12, which can also be used to draw conclusions about their state of health.

The thermal imaging camera can also be used to display a scantily clad person 2 in a true-to-life manner by means of the temperature difference. The tiltable couch also makes it possible to present the examined person 2 in several different positions, either almost sitting or almost lying down, which increases the accuracy of the measurement. The measurement accuracy is important because the length and cross-section of the body segments are included in the evaluation of the impedance measurements. This way, the results of the non-contact measurement of the person can be used with the other physical, especially electrical, data in joint equations or a set of equations to obtain the greatest accuracy.

The two- or three-dimensional representation could then be used to identify the electrodes that are to be used for ECG recording, impedance spectroscopy, impedance plethysmography from the multitude of electrodes. This is particularly important if so-called EASI leads are to be used according to Dower (J Electrocardiology Suppl. 1988, p. 182-187), in order to reconstruct the conventional leads according to Einthoven, Goldberger and Wilson from these leads, in particular via regression equations, via the xyz model or using artificial intelligence or neural networks.

Of course, a swivel arm can also be used in a known manner with cables and electrodes attached thereto (not shown). In this case, the electrodes would preferably be attached to a common belt, which, for example, is placed on the chest of the person and contact to the examined person 2 is for example produced only by gravity or by an elastic rubber belt or also by actively deformable areals, such as inflatable chambers/blisters.

FIG. 2 shows a detail of the inventive diagnostic system 1, which comprises at least three contact electrodes 17 to affix a chest belt 13 and by which electrical signals can be transmitted to a chest belt 13. Here, too, no cabling is required; contact can instead be made by positioning a person 2 to be examined on the diagnostic system 1, which is also here formed by means of a swivel recliner. This person 2 can thus optionally engage a chest belt 13 provided on the diagnostic system 1 unassisted such that the diagnostic system 1 can continue to be operated without additional personnel.

FIG. 2 thus shows how the support surface 11 could be embodied with a simultaneously used chest belt 13. The electrically conductive electrodes -Eᵣ-, -Aᵣ, -Sᵣ, -Ir- or -Eᵣa-, -Aᵣa-, -Sᵣa-, -Iᵣa- in FIG. 1 applied in the upper body region 6 can be replaced or supplemented by the chest belt with the chest wall electrodes, which make electrical contact with the electrodes on the back side of the chest belt 13. An electrode could be provided for an electrical contact for grounding, an electrode could be provided for central terminal of Wilson (short-circuited electrodes of positions I, II and III according to Einthoven), a further electrode could be provided for establishing the electrical contact to at least one chest wall electrode, preferably V4.

The bar 29 can be removably mounted in the bar bracket 38, which is particularly necessary when using a mat as a support and bracing surface.

FIG. 3 shows the inventive diagnostic system 1 with a person 2 to be examined. As can be seen, person 2 has donned a VR headset 14, by which for example information about the state of health and the status of the examination performed can be displayed to person 2. For example, during the examination a visualization of his/her body can be displayed to the user via the VR headset 14, said visualization showing how far the examination has already progressed and what data has already been determined. The state of health of the individual body regions determined in this way can be color-coded in the visualization. This VR headset 14 can also be used for operation; a screen can therefore be omitted; the keyboard can also be omitted if voice control is used.

Furthermore, a breathing tube 39 is shown, which is connected to the VR headset 14. If the patient or a person 2 to be examined breathes through the nose, the respiratory volume, the respiratory graph and the composition of the respiratory air, in particular CO2, O2, and volatile gases, such as acetone in particular, and the like, can be measured; as a result, the lung function and metabolism can be recorded without additional effort.

FIG. 3 further shows that a neck roll 10 is provided in the neck region 7. This neck roll 10 consists of elastic material or actively expanding areals and, like the arm regions 4, the leg regions 5 and the knee regions 40, comprises preferably band-shaped electrodes. The electrodes in the leg regions 5 and knee regions 40 are particularly suitable for detecting artery and vane circulatory disorders of the leg by means segmental plethysmography or of the lower leg by using the electrodes in the knee region 40. For this purpose, additional compression cuffs, e.g., also U-shaped on the lower legs (possibly also circular, not shown), which also permit determining an ankle brachial index ABI. For this purpose, the methods of reperfusion and venous occlusion plethysmography are known, wherein volume changes can also be recorded. Particularly in all joint regions, the support surface could be mechanically actively adapted by actuators to the joint movements corresponding to the joint axes, thus also permitting physiotherapeutic measures.

Furthermore, FIG. 3 shows a chest belt 13 with which an ECG can be generated. This chest belt 13 is connected to the diagnostic system 1 according to FIG. 2 on the back with contact electrodes 17 concealed by the body, thus eliminating the need for cabling as required in conventional devices for generating an ECG.

As shown in FIG. 3, the examined person 2 can as desired also observe the results of the recording with a personal screen mounted above the recliner or with a headset, for example in the form of a virtual reality headset 14. Using headphones 30, the person 2 can also acoustically follow the procedure of the examination with the explanations or also can also receive instructions. When using a screen, it is advantageous to affix it in such a way that undesired observers cannot observe the displayed personal data of the person. If person 2 can track the collection of biological data in real time or in a timely manner simultaneously or possibly only with a short delay during the recording process, this also increases the motivation to have this examination performed.

The swivel recliner is preferably also positioned on a scale 41, preferably a surface scale or one or more load cells, so that the weight of the person can be determined automatically.

Between the examinations, the examined person 2 or auxiliary personnel can clean the person's support surface 11 with a cleaning wipe or cleaning spray. Preferably, a cleaning wipe or spray is used for this purpose, which simultaneously also serves as electrode contact liquid. For this purpose, a saline solution, for example, similar to a physiological saline solution, especially physiological saline solution, could be used. This cleaning wipe or spray should be both bacteriostatic and virostatic, especially against coronaviruses, and should be soaked with the corresponding chemicals, for example with 70% 2-propanol or 80% ethanol. There could also be a disposable spread made of paper or other disposable material for the parts of the support surface 11 that are not used for electrical contact. Thus, the complex diagnostic system 1 is ready for examining the next person.

FIGS. 4 and 5 show a detailed illustration of a support surface for arms or legs that can be used in the arm regions 4 and the leg regions 5, for example. In order to accommodate all feed lines to the electrodes in a protected manner, the support surface must be embodied in two layers, and thus comprises a near-body support surface 11 that can be made of washable plastic, for example, and a remote-body bracing surface 19 that can be made of a durable fabric, for example. This bracing surface 19 can also be made of durable plastic fabric for the torso of the examined person 2 or, as shown here, can be made of hard material for the electrodes 3 on the arm regions 4 and the leg regions 5, in particular plastic or metal. Electrical lines and electronic components such as electronic switches, pressure sensors 44, etc. are arranged here between the support surface 11 and the bracing surface 19 such that they are not visible to a user.

The near-body support surface 11 can be embodied to be deformable. In the design example, a deformable intermediate layer 20 is arranged under the support surface 11. This intermediate layer 20 can, for example, consist of foam or of another deformable medium, such as a fluid, a gas or a liquid, which can also actively expand by changing the pressure. By changing the pressure in this intermediate layer 20, the near-body support surface 11 can adapt particularly well to the human body. Sensors, such as pressure sensors 44, light sensors 42, sound sensors and/or energy sources preferably located opposite thereto, such as light sources 43, in particular LEDs, could thus press particularly well against the body and thus ensure a good transmission of the pulses or signals into the body. In addition to electrical properties, mechanical, optical, and acoustic properties of the individual parts of the body can thus be analyzed

If—as shown schematically in the exemplary embodiment—a pneumatic pressure source 21 and a pressure measuring device 22 for measuring the pressure are present in this intermediate layer 20, the blood pressure can thus be determined by oscillometry and/or auscultation, and/or a continuous pulse and pressure analysis can be carried out. As is known, the Korotkoff noise and the time of the appearance and disappearance of a certain sound level are used for the auscultatory measurement, the registration of the pulse amplitudes at different pressures for the oscillometric measurement, and the pulse amplitudes at the time of the relaxed arterial wall for a continuous pressure measurement, provided the blood pressure in the artery and in the compression medium is the same. All of these methods can be achieved with the diagnostic system. The method of continuous blood pressure measurement can then be used particularly well if the amount of blood within the compressed arterial section is for example measured optically, for which the light source 43 and the light sensor 42 are well suited. For this purpose, the support surface (here: U-shaped) does not have to be closed, and a corresponding pressure can nevertheless be applied to the forearm or lower leg because the extremity is bound by bone at this location at the exposed location. The body part can thus also be simply inserted into the U-shaped support surface at this point without further manipulation. In order to prevent the arm from being pushed out of the U-shaped support surface when pressure is increased, a pressure-free zone 18 could alternatively be provided on the bottom of the U, and in which no pressure is exerted because a bone boundary is provided by the ulna in this case as well. In this embodiment, the U-shaped support surface is thus at least partially divided into two parts, wherein both parts are pressurized with the same pressure. The same pressure does not have to be built up in the two parts of the cuff; different pressures could also be advantageous, e.g. to achieve the most intensive contact possible with the artery. The at least two-part arrangement is a possible embodiment; of course, an integral embodiment is also possible, as shown in FIG. 4. For this purpose, the parts of the support surface that do not come into contact with the body or the bracing surface 19 should be designed as pressure-resistant as possible so that the pressure is primarily applied only to the human body. It goes without saying that in the event a liquid or gas is used as the medium, a hydraulic pressure source 21 such as in particular a pump can of course also be used as the pressure source 21.

FIGS. 6 and 7 show a chest belt 13 for the inventive diagnostic system 1, which is embodied for particularly precise recording of an electrocardiogram, for example. FIG. 6 shows a front side of the chest belt 13 and FIG. 7 shows a rear side of the chest belt 13. Electrical contacts are provided on the rear side of the chest belt 13; said contacts correspond to contact electrodes 17 on a support surface on a corresponding region of the diagnostic system 1 or the recliner as shown in FIG. 2 in order to be able to transmit electrical signals from the support surface to the chest belt 13. The chest belt 13 can be applied unassisted by a person 2 to be examined such that an electrical connection of the chest belt 13 to the diagnostic system 1 is possible without a plug or the like.

The chest belt 13 comprises on the inner side a plurality of chest wall electrodes 23 with which so-called Wilson's leads can be determined. The central terminal Wilson can be corrected accordingly by means of appropriate algorithms. The individual chest wall electrodes 23 can be switched on and off by means of a multiplexer. A closure, for example a hook & loop closure, a buckle, a snap device 24 or the like, is arranged on the front side of the chest belt 13 such that a person 2 using the diagnostic device can easily fasten the chest belt 13 unassisted. An adjustment device can also of course be provided in order to easily change the length of the belt and to be able to easily adjust the belt to different size ratios in this way. The chest belt can also comprise one or more expandable chambers outside the electrodes; said chambers actively press the electrodes onto the body, wherein the quality of the signals can control the contact pressure.

As an alternative or in addition to the chest wall electrodes 23, the chest belt 13 can naturally also have electrodes at positions E, A, S and I according to EASI in order to be able to make so-called vector-cardiographic and EASI leads according to Dower (J Electrocardiology Suppl. 1988, p. 182-187; Dower G E, Clin. Cardiol. 3, 1980, 87-95).

A simple variant of the chest belt could also be provided as a tensioning strap 6, e.g., elastic or also non-elastic equipped with a tensioning device 47, wherein the tensioning strap is fastened to the support surface or the bracing surface, for example. The examined person tensions said strap across the lower part of the chest, for example, and fastens it on the vis-à-vis side, for example, with a snap device 24. Good electrode contact can be ensured if this tensioning strap is elastic or comprises a tensioning device 47. When the tensioning strap is accordingly fastened, e.g. to the support surface or bracing surface behind the body, said strap creates good electrode positions around the body, which ensures numerous possibilities of electrode positions.

In order to be able to carry out the EASI leads on persons with a small and large body circumference, it is recommended to equip the chest belt or a tension strap with a plurality of electrodes, of which those are then activated that correspond to the EASI positions. To determine the correct electrode positions, either the ECG signal itself or the 3-D representation of the person can be used, especially if the electrode positions can be identified on the outside of the belt by markings, e.g. color coding.

For example, the tensioning belt could be fastened to the side of the support surface behind the patient approximately at the position Iᵣ or Aᵣ of the contact point of the bracing surface and could then wrap behind the patient clockwise or counterclockwise along the support surface of Iᵣ in direction Aᵣ and subsequently in front of the body in the direction of the starting point in direction Iᵣ or vice versa, where said tensioning belt is closed, for example, with a snapping device 24 as used on a seat belt. In this embodiment, the tension spring is best integrated into the snap device 24 such that the fastening of the belt to the bracing surface and/or the support surface is very easily implemented, for example, along a seam location 48 by a seam, or glued or welded. At this seam location 48, the electrical connection to the electrodes Iᵣ, Mᵣ, Aᵣ, A, E and I, to the chest wall electrodes and/or any other existing electrodes can also be established. As is known, the tensioning device could be carried out, for example, by a roller tensioned with a spring and locking mechanism, as is known from many window roller blinds. A loop for guiding the tensioning belt has shown to be effective, for example, approximately in position Aᵣ of the support surface such that the further direction of the tensioning belt progression is predetermined. A complete or almost complete encirclement of the body with the tension strap is thus possible. Thus, all positions, both the classic positions I, E, A of the EASI leads on the front of the body as well as the Iᵣ, Mᵣ und Aᵣ positions, as well as the positions of the chest wall electrodes, if necessary, can be equipped with electrodes on the inner side of the tensioning belt. The position S of the EASI leads could, for example, also be accommodated as a forehead electrode 45 on the forehead in the edge of the VR headset. Given a corresponding tensioning force of the tensioning belt, interferences of the electrical signal with artifacts and electrical noise during body movements are thus prevented.

A correspondingly high spring force in the intermediate layer 20 has also shown to be effective on the U-shaped support surfaces for arms and legs such that the increase of the contact pressure can either be achieved by increasing the pressure in the fluid or also by mechanical spring force. Manufacturing all electrodes, not only said chest wall and EASI electrodes, but all electrodes of the diagnostic device from silver chloride or stainless reduces the electrode potential and the noise of the signal. This is particularly important because it is beneficial to hang the discovery of the impedance plethysmographic signal on the R-wave of the ECG, which is why a stable R-wave detector in the software is of great advantage. Thus, all segmental as well as whole-body impedance plethysmographic signals can then be carried out and evaluated very precisely, e.g. by infeeding and reading the impedance between one or both arms, and one leg or both legs. From these signals, together with the determination of the start of the propagation and the valve closure, the stroke volume and the cardiac minute volume can then be determined in a known manner with the help of the basic impedance zO of a calibration factor, the duration of the cardiac cycle, the duration of the largest amplitude of the impedance plethysmographic signal until the start of the next cardiac cycle. The calibration factor is calculated from height, electrical resistance of the blood, hematocrit, electrode distance and height (see e.g. Koöbi, Intensive Care Med (1997) 23:1132-1137). The impedance plethysmographic signals of the whole body and/or body segments, e.g. of the chest, abdomen, arms and legs of several to many heartbeats, are superimposed and a template is formed. The characteristics such as amplitudes, high amplitude pulse, low amplitude pulse, width, shape, etc. are determined from this. These templates could, for example, also be formed separately for inspiration and expiration, since inhalation and exhalation can also be detected via the impedance plethysmographic signal and/or via the 3-D headset with breathing tube. The impedance plethysmographic signals of the whole body and chest, arms and legs can then also be used to calculate the pumping force, the detection of cardiac insufficiency, of the pulse wave time or volume wave time, the calculation of the performance under stress, with or without use of extracellular water and/or use of the calculated muscle mass and/or the calculated fat mass, with or without use of the calculated abdominal fat and other parameters. Extracellular and whole body water and the water of the segments result from the resistances of the body and its segments at low (e.g. 5 kHz) and high frequencies (e.g. 500 kHz), since high but not low frequencies can penetrate the cell membranes. An accumulation of extracellular water is known to be a sign of heart and/or kidney weakness, liver diseases, thromboses, lymphedema or dysproteinemia. Multiple regression equations, a discriminant analysis or similar methods or even artificial intelligence, such as neural networks, can also be used to determine the water compartments. The additional use of the contactless measurement of the person in combined equations increases the accuracy. In addition, “gold standard methods” such as ergometry, echocardiography, whole body DXA, double gas breathing method or CO2 rebreathing, biochemical markers such as BNP, can also be used for this purpose.

This allows all twelve leads of the standard ECG to be reconstructed with a few electrodes or a portion thereof. In order to achieve position V1 and V2 according to Wilson or position S according to EASI with a single chest belt 13, the chest belt 13 can also on the chest side of the examined person 2 have an extension 26 in the direction of the manubrium of the sternum of the examined person 2. This extension 26 can also be pressed against the examined person 2 by levering. For example, a small housing 25 can be arranged above the sternum, which housing, for example, presses the chest belt 13 against the examined person 2 by means of a spring located behind said housing. Signals are usually amplified with electronics located in the housing 25 and can also be transmitted wirelessly, for example by means of Bluetooth, to a measuring module with CPU 36 attached to the diagnostic system 1 in order to evaluate the signals and to infer diagnostics.

FIG. 7 shows the rear view of the chest belt 13, wherein the chest belt 13 carries a plurality of chest belt outer electrodes 27 on the outer side, i.e. on the side facing away from the body, which outer electrodes can make contact with the contact electrodes 17 of the support surface in the upper body area 6, which contact electrodes are shown in FIG. 2. The chest belt 13 shown thus carries electrodes on the inner side, namely the chest wall electrodes 23 shown in FIG. 6, and also on the outside, namely the chest belt outer electrodes 27. In addition, an additional extension 26 directed downward is also provided on the rear. Using this extension 26, the chest belt outer electrodes 27 can then be brought into contact with the contact electrodes 17 of the support surface 11 even when the examined person 2 wears a brassier. The chest belt 13 attached to the examined person 2 is preferably an at least partially elastic chest belt 13; in particular in the areas of the chest belt 13 that do not carry any chest belt outer electrodes 27 or chest wall electrodes 23, the chest belt 13 can be adapted to be very elastic such that the electrodes can actually come into close contact with the body.

This chest belt 13 can thus also be worn during physiological interventions, such as a tilt table examination, without the electrodes being able to shift. If the examination lounger is embodied as a tilting chair, hemodynamic measurements can also be performed with this device after a change in position.

In order to compensate for different body dimensions, a plurality of chest belts 13 are provided, wherein, for example, the examined person 2 is held in position with the aid of calipers, which chest belt 13 matches the body size of the examined person 2. This means that electrical contact with the person is established without the use of personnel.

The chest belt 13 is generally not mandatory. Thus, a diagnosis can already be made with electrode leads according to Einthoven and Goldberger or with the Eᵣ, Aᵣ, Sᵣ and Iᵣ leads, which are possible with the electrodes in the support surface 11.

The design of the diagnostic system 1, as described, is of great advantage in times when labor represents the main cost factor.

A version of the complex diagnostic system 1 is also possible, wherein the person is, for example, is in a leaning position. The arm electrodes should in this case also advantageously either be embodied as half-shells into which the person places their forearms, or also as bands on the wrist. The electrodes in the leg regions 5 can in principle also be arranged on the sole of the foot or on the ankle, for example by the person standing on said electrodes, or also by wearing half shells on the calves. However, measurement electrodes have the disadvantage of a high series resistance. For example, a scale could then also be used as a standing surface to determine the weight of the person at the same time.

The person can also be rendered and sized in 2-D or 3-D by using an imaging device. Using this version of the complex diagnostic system, the person could then also be examined in a horizontal and also in a nearly vertical position, whereby the autonomous and hemodynamic integrity of the person 2 can be determined at the same time. In addition, all hemodynamic parameters and fluid parameters and their change can be determined by orthostasis.

FIG. 8 schematically shows a section of an electrical circuit of the diagnostic system 1. In this case, a body part of a person 2 examined with the diagnostic system 1 is shown as impedance 32 (consisting of its subcomponents, like a real part, imaginary part and phase angle), which impedance 32 connects two electrodes, for example a feed electrode 8 in the arm region 4 and a feed electrode 8 in the upper body region 6 such that the impedance 32 can be detected via measurement electrodes 9 not shown here, in that an electrical current is fed through the feed electrodes 8 with a power source 35 and a voltage drop between the measuring electrodes 9 can be measured with the measuring electrodes 9 mapped to the respective feed electrodes 8, which voltage drop is dependent on the impedance 32. The subcomponents of the impedance, namely real resistance, apparent resistance and phase angle, can also be used to calculate the diagnostics.

Switches 31 provided here directly adjacent to the feed electrodes 8 can be used to switch off the feed electrodes 8 by downstream electronics and the patient connection lines. Switching off electrodes that are not needed or not activated prevents any influence of the cables or lines 34 of inactive electrodes on the measurement result.

Furthermore, switching devices 33 are provided close to the power source 35. The switches 31 arranged near the feed electrodes 9 and the switching devices 33 near the measurement module with CPU 36 are usually actuated synchronously. The patient connection lines are embodied as shielded lines 34, wherein a so-called “Shield Driver” controls the potential of the shield based on the potential of the line 34. On currently active electrodes, any unwanted current flow caused by insulation resistance and coupling capacity of the inner conductor shield is prevented.

The measurement module with CPU 36 can in this case contain circuits for multichannel ECG, impedance feed and impedance measurement, multiplexers, one or more evaluation units for contact data and non-contact data, etc. and is preferably mounted as close as possible to the examination table or the diagnostic system 1.

The power source 35 is preferably connected to the feed electrodes 8 via shielded lines 34 in order to be able to forward to the CPU and evaluate data about the determined impedance 32.

The power source 35 should be embodied as a bidirectional power source in order to be able to detect the respective alternating current resistance, i.e. the impedances 32 of individual body parts of the person.

The ECG circuit and other circuit parts, such as for further physiological signals, are not shown in FIG. 8 for easier comprehension.

A further schematic diagram of the diagnostic system 1 is shown in FIG. 9. Here, two galvanically separated power supplies 35 are provided, which can be connected to feed electrodes 8 at different positions of the support surface, as shown schematically. The dashed lines indicate possible connections and activated connections are represented by the solid lines. As can be seen, the left power source 35 is thus connected here to a feed electrode 8 in a left arm region 4 and to a feed electrode 8 on a left leg region 5 such that current can be applied on body regions between this feed electrode 8 with the power source 35 in order to determine the impedance 32 of this region.

The right power source 35 is accordingly connected to feed electrodes 8 in a right arm region 4 and an upper body region 6 such that the impedance 32 of the upper arm shown on the right in FIG. 9 can be determined with this power source 35 or associated measuring electrodes 9. Due to the galvanic separation, measurements that do not influence each other can be carried out with the two feed electrodes 8. More than two power supplies 35 can also be provided.

These modules could come as close as possible to the recliner or the integrated electrodes. For the sake of simplicity, only two galvanically isolated power supplies 35 are shown; their number can be increased as desired. As shown in FIG. 9, for example, the entire body and also the left arm can be measured. As indicated with the dashed lines, every other segment could also be measured depending on the electrode position; here, for example, the dashed lines show the measurement of the thoracic segment and left lower leg.

As shown in FIG. 10, it can also be provided to embody the power supplies 35 as matched power supplies (both positive and negative). However, it can alternatively also be provided to use regulated power supplies, which means that an elaborate adjustment of the power supplies can be omitted.

For this purpose, FIG. 10 shows one of the possible embodiments, wherein the offset of the two power supplies 35 can be controlled in reference to one another by an analog electronic circuit or by a digital controller (in a microcontroller). For example, the common mode potential of the patient can also be used as a control variable. This common mode potential is usually measured anyway for ECG recording with the N electrode on the right leg (RLE), whereby this electrode can also be used as a measuring electrode. This then also reduces the number of necessary electrodes. The corresponding circuits (see e.g. https://en.wikipedia.org/wiki/Driven_right_leg_circuit) can thus be actively compensated by the right leg drive electromagnetic interference, e.g. also network hum.

The AC resistance to be measured is marked with z1, the measuring electrodes with EL in the dashed frame, the unused electrode with EL outside the frame, D/A stands for digital-analog converter and CPU for central processing unit with measuring module. For the sake of simplicity, the additional measurement electrodes are not shown in this image.

FIG. 11 shows a circuit arrangement for carrying out an electrode contact measurement in order to ensure sufficient electrode contact and also to select from the plurality of electrodes the one that has the lowest contact resistance to the examined person 2.

An input amplifier 37 and a shield driver are provided for the measuring electrode 9, labeled “V”. However, the electrode contact measurement reduces the input resistance, which can therefore not be carried out simultaneously with the current measurement, but is preferably switched off during the same measurement.

Other electronic circuits from the prior art are also known for the contact measurement, which could also be used in the inventive diagnostic system 1.

Alternatively, a measurement current could also be fed in, as shown in FIG. 11; pressure sensors 44 could also be used for this purpose, whereby, for example, those electrodes arranged close to pressure sensors and on which a greatest contact pressure is measured could be used for the measurement.

FIG. 12 shows a three-dimensional image of a person 2 to be examined that was acquired with the inventive diagnostic system 1. This image can also be captured on a dressed person 2, in that a contour of the person 2 is recorded three-dimensionally, in particular with infrared cameras and the like. The contour can firstly be used for rendering in the VR headset 14. In addition, the contour can also contribute to the determination of a state of health and in particular provide information about a waist-to-hip ratio and a body mass index or the like, or can contribute to the formation of these values.

Measuring points 28 are also drawn here on both ankles, in the step, on the Xiphoid and on the upper edge of the sternum, which measuring points are preferably recorded automatically, in particular with a camera 12 arranged above the person. These measuring points 28 are advantageously used to determine the calculations of the body compartments with the aid of the multifrequency impedance based on the segment lengths. The arms, not shown here, can also be represented by their length, diameter, and configuration.

FIG. 13 shows a corresponding image, which can also be displayed in a VR headset 14, for example. Such an image can also be embodied as a glass image of a body of the examined person 2, for example by representing a beating heart, pulsating arteries in the body's own rhythm, furthermore the breathing and other parameters (referred to as “etc”). Corresponding data can be acquired in the support surface with sensors, as discussed.

If pathological changes are observed, they can also be displayed in real time or in a timely manner, for example by flashing or changes in color.

In combination with the representation of the human body in 2-D or 3-D, the location of any problem zones can then be graphically displayed to the examined person 2. The online presentation of the changes during recording is also particularly advantageous because it can be used to motivate the patient to have this examination performed. For example, in the three-dimensional figure obtained by means of a 3-D measurement, the heartbeat, the activity of the vessels such as heart rate, blood pressure, aortic stiffness, cardiac output as well as numerous other parameters, the circulation of the organs and all other biological data obtained can be displayed in a two-dimensional or three-dimensional manner. For example, a flashing online representation of conditions that are not in an optimal range, such as lack of muscle mass, excessive fat mass, pathological changes in organs, such as impaired heart function due to cardiac insufficiency, excessively stiff aorta or lack of blood flow to the legs could be indicated by coloring or otherwise indicated online.

Columns pulsing in the heart rhythm could indicate heart rate, blood pressure level, aortic stiffness, cardiac output and other physiological parameters. This could then motivate person 2 to change his lifestyle accordingly in order to halt or reverse pathological conditions.

At the same time, the examined person 2 could also be equipped with headphones 30 or speakers, preferably close to the ear, in order to only inform the examined person about the progress of the examination, in order to possibly receive instructions about the correct posture, or also a request for the actuation of switching operations or information about the termination of the examination.

The other illustrations shown are examples of the use of the graphical representation of biological or biochemical data obtained from the patients.

FIG. 14 shows a bar diagram with an illustration of the individual body functions created with the inventive diagnostic system 1, for example a bar chart and simultaneous representation of human figures, which either correspond to the figures as recorded by contactless measurement, or only—if sufficient—as a symbolic representation of the body to indicate findings that deviate from the standard by graphical labeling, for example, gradient, redacting, or coloring.

This figure shows an exemplary embodiment for the depiction of cardiac output, cardiac wall tension, hydration, perfusion of the legs with depiction of a possible peripheral occlusive disease, pulse wave time, physical performance, total body water, depiction of overhydration or underhydration in the area of extracellular water, appendicular muscle mass of both the upper and lower extremity, fat mass, and torso fat mass. The individual parameters are indicated in the label.

The results are shown firstly in the form of a bar chart, wherein the width of the bar represents the normal range. The position of a possibly colored marker shows whether the corresponding parameter is in the normal range, below the normal range or above the normal range. Secondly, the right side of the figure shows a human figure, whereby individual properties of the human body are shown only in a separate figure. Thus, it can be shown separately whether, for example, the cardiac output, the vessels, the circulation of the legs, the muscle status, the body fat, the autonomous nervous system are in the normal range, or whether there are moderate or severe changes that are for example indicated by gray coloration or black coloration.

In order to make the representation even easier, the corresponding parameters are also shown in the form of human figures, whereby a special color, for example a white coloring of the figure, means that all collected parameters are in the normal range. Different coloring, such as graying or black coloring of the individual organs or even yellow or red coloring or another color, shows whether the physician must pay close attention to a certain examination result and further examinations must be carried out in this regard.

This figure firstly shows the heart, and secondly the major vessels, such as the artery, the muscle mass, the fat mass, the body fat mass, the arterial circulation in the individual figures.

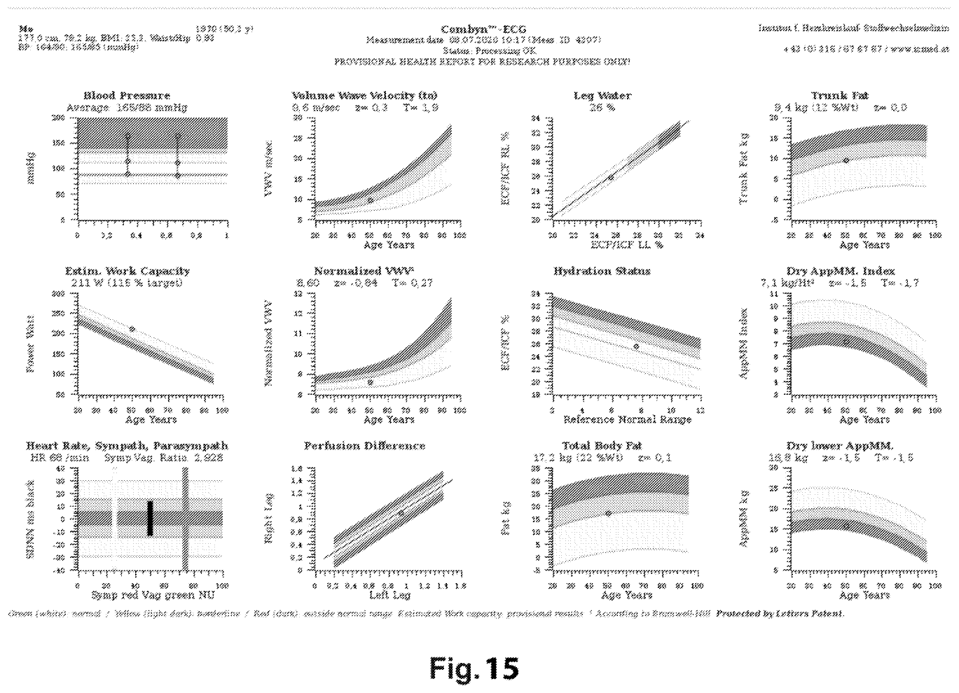

FIG. 15 shows an age-based standard diagram created with the inventive diagnostic system 1, in which the individual measured values of the person are drawn by a measuring point 28 in nomograms with the age-appropriate standard values. The nomogram graphs shown here correspond to the age-adjusted physiological range; any measurement outside this physiological range is indicated by a measuring point 28 outside the bar. The optimal ranges for the graphs are displayed here, for example, by green coloration of the age range, limit ranges by yellow, and clearly pathological ranges in the color red.

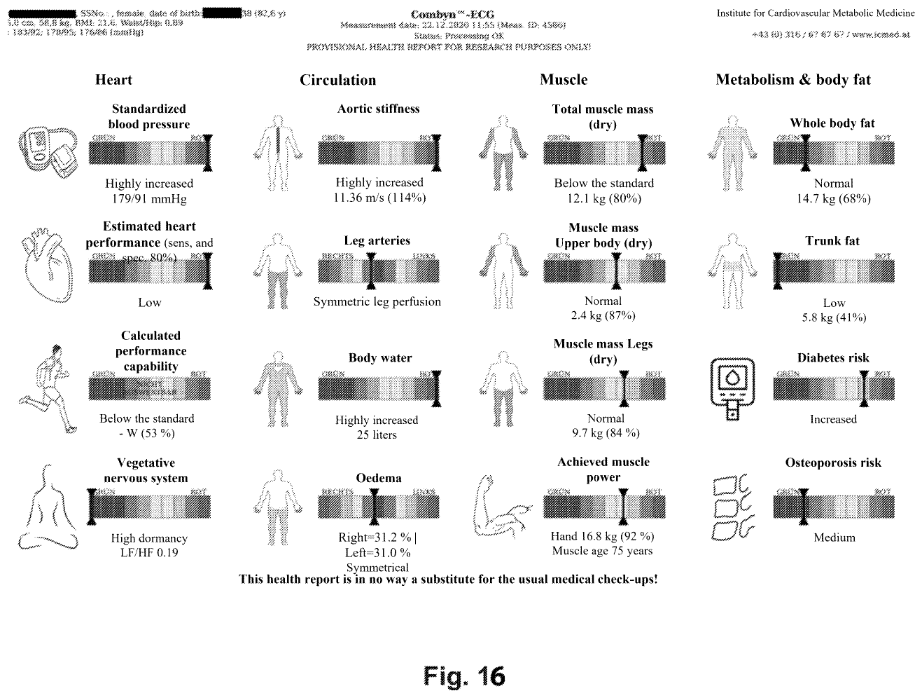

FIG. 16 shows a health report created with the inventive diagnostic system 1, in which the measured values are displayed in the form of traffic light colors green, yellow or red in comparison to the age-appropriate collective, wherein the color green indicates satisfactory health values, the color yellow shows limit values, and the color red shows values in an unfavorable range. Naturally, other colors can also be used. The individual states and functions shown are also marked with generally understandable symbol images.

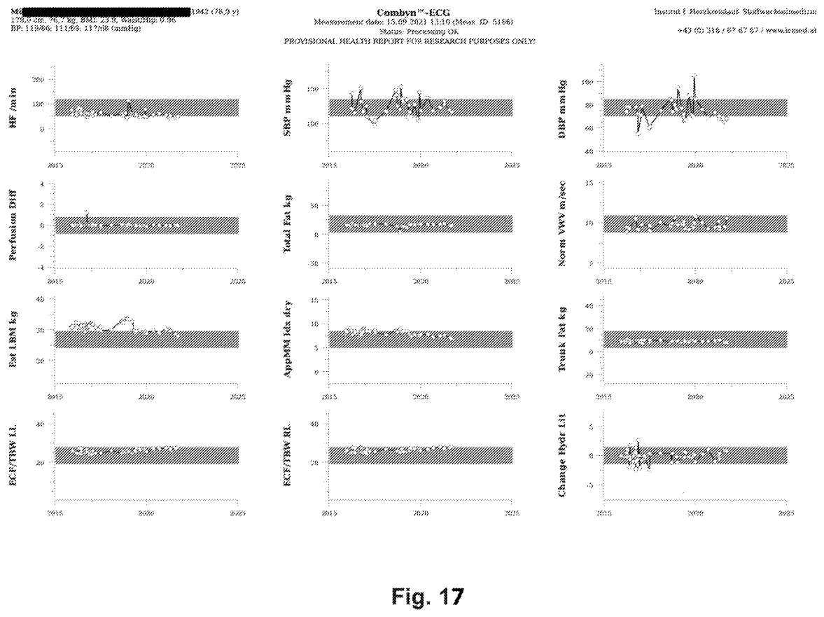

FIG. 17 shows a plot of the measured values created with the inventive diagnostic system 1 when the examined person 2 participates in this investigation more than once. The individual measuring points 28 are connected by lines, so that more favorable or unfavorable changes can be represented at a single glance.

The illustration above has proven particularly effective when for example used in combination with already known medical data, such as laboratory chemical tests, possibly obtained from point of care measurements, the EMG, temperature measurement, oxygen measurement, nerve conduction velocity, computer tomography, magnetic resonance, ultrasound, X-ray, endoscopic examinations.

The examined person 2 can then immediately transmit their own findings online via Bluetooth, preferably by means of color printouts.

In order to facilitate administration, the examined person 2 could also read in a payment confirmation, a ticket, for example via a bar code, which he/she was given in advance in order to obtain authorization to carry out the examination.

It is advantageous if data and diagnoses recorded with the inventive diagnostic system 1 are stored in a database such that a person 2 can easily understand a performance of their state of health when said diagnostic system is used several times.

It is particularly advantageous if the automated measurement of electrical and, if necessary, other values of a human body are combined with the inventive diagnostic system 1 based on a structured questionnaire.

This questionnaire is preferably carried out in the form of a dialog tree, whereby the answer to a question results in another question resulting from the answer to the question. This questionnaire can be done using a language program or also in the form of a preformatted form, preferably in digital form, in which corresponding answers can be entered by checkmark or redacting.

In order to make it as easy as possible for the user to answer this questionnaire form, the questions could be executed such that they can only be answered with yes or no or by entering a number. This questionnaire could also be answered during the examination or only after the examination, in particular also online. The results of this questionnaire will then be linked to the collected biological data, for example by an expert system.

This data can be transmitted in a known manner to a central database, preferably when using many of these complex diagnostic systems 1 to a central database, which stores the measurements of many deployed complex diagnostic systems 1, preferably anonymously.

For example, automatic computer systems could also transmit remote suggestions for optimizing the health of the examined person 2 or suggest a visit to a physician or a health facility in the event of pathological findings. Additional online databases can also be used for this purpose so that the data can be linked to other biological data. For example, laboratory data, performance data such as walking speed, kilometers traveled, altitude meters, calorie consumption and the like could then be linked to the data obtained here in order to permit even better information about health and even better health advice.

The diagnostic system 1 is also suitable for communicating information about respiration, metabolism, especially sugar metabolism, liver and kidney function as well as blood counts, in addition to cardiovascular parameters and body composition. For this purpose, appropriate sensors can be integrated into the diagnostic device.

For example, accelerometers or Microphones integrated into the support surface, for example at the location of the EASI electrodes, are well suited for monitoring respiration; for example, the breathing sounds, respiratory rate, respiratory depth, duration of inspiration and expiration can be compared for each side and used to detect bronchial asthma, asthma cardia, pulmonary congestion, pulmonary infiltrates such as pneumonia, one- or two-sided pleural effusions. The breathing sounds change, for example, from vesicular breathing to intensified breathing, dry rattling sounds, moist rattling sounds, weakened breathing sounds, etc. The impedance plethysmography signals can also be used to detect the duration of inspiration and duration of expiration by changing the base impedance with inspiration and expiration, and also for an even better diagnosis of respiratory disorders and cardiac insufficiency, also by prolonged expiration and dry and/or moist rattling noises, wherein cardiac insufficiency can also be detected with the diagnostic unit using Cheyne Stokes breathing.

Furthermore, all known methods for measuring blood sugar (see for example Clinical Chemistry 1999, 45:2 165-177) can also be integrated into the diagnosis system 1; other techniques such as skin permeation methods could also be used (Chuang, J. Diabetes Science and Technology Volume 2, 595, 2008) or Graphene Thin film technology (Lipani L. Nature Nanotechnology, 2018, vol. 13, no. 6, pp 504-511).

A disorder of sweat gland secretion can be detected by recording the function of the sweat glands, such as in the Sudoscan (Casellini C M, 2013, Diabetes Technology & Therapeutics Vol 15, 948). To do this, the palms of the hands and feet should be brought into contact with stainless steel plates, for example, through which a low current of a few volts flows. For example, the steel plates can be pressed against the exposed hand and foot using springs. Sweat secretion can then be recorded via the electrical measurement of chloride ions. This can be disturbed not only in diabetes, but also in prediabetes. In this way, a unilateral disorder can also be differentiated from a bilateral disorder. Ethanes and n-pentanes concentrations have potential for the detection of oxidative stress.

Furthermore, the camera 12 can also be used to measure kidney function, in particular by changing the color of the skin, such as through urochromes, or changes in liver function through icterus, which can be detected by changing the color of the skin and sclera using the non-contact display.

Furthermore, a vibration mechanism can also be integrated into the support surface 11, which is positioned in the part of the support surface that is in contact with the liver. In a manner known from the prior art, a “vibration scan” using ultrasound can then be used to determine the traveling speed of the pulses emitted and thus the hardness of the liver and therefore its connective tissue and/or fat content. Transient vibration is particularly useful here, because the sound and ultrasound emitted and received can be separated in time.

On the one hand, acoustic or elasticity waves can be analyzed. This allows chronic and acute liver diseases, such as fatty liver or chronic hepatitis and cirrhosis, to be detected. On the other hand, ultrasound can be used to determine the bone density of the heel or metacarpal bones, for example, and diagnose osteoporosis or the risk of fractures. Both the broadband ultrasonic method, which assesses the frequency dependence of the ultrasonic attenuation, and the SOS method, which uses the speed of ultrasonic propagation, can be used for this purpose. A fluid feed in the form of a deformable bladder could be an advantage. Thus, the diagnostic system 1 according to the invention can also have vibration devices and sensors with which vibrations can be applied specifically to individual body regions and mechanical reactions of the body to these vibrations can be measured.

Anemia can also be detected by optical methods, for which sensors on the eyes or extremities, especially the wrist and fingers, are particularly suitable. An erythema index can be determined there using a camera 12 (Collings S, PLOS One, 2016 Apr. 12; 11 (4): DOI: 10.1371/journal.pone.0153286), for which the capillary bed of the nail fold is particularly suitable.

For example, the hemoglobin can be determined with sufficient accuracy on the radial pulse using a multi-wavelength pulse oximeter (Dreyfus J, Annals of Emergency Medicine Volume 57:330 2011).

For all applications in which the fingers are examined, an additional finger cuff or glove in which the sensors are built in, which can not only keep out stray light, but also establishes particularly good skin contact regardless of the finger diameter, especially with an elastic design, proves its worth. Optical detection of iron deficiency by measuring zinc protoporphyrin, for example on the lip, wrist or finger, or other methods (Hennig G, Nat. Commun. 2016, 7:10776 doi: 10.1038/ncomms10776) are also possible, which is so important in view of the widespread iron deficiency worldwide.

To capture the details of the face, the planned 3D headsets should also be equipped with additional sensors such as optical sensors. Not only the capillaries of the mucous membrane, but also the color of the sclera can be measured and assessed. For example, haemolysis or liver disease can be detected by an increase in bilirubin, and a vitamin B12 deficiency can also be detected, in which the skin is also characterized by paleness and a dirty brown colour. For this purpose, constant illumination in terms of both intensity and color tone is favorable, which is made possible by a standardized and calibrated light source 43 at least for part or parts of the body.

The person 2 to be examined can also be equipped with a breathing mask to record the amount and speed of inhalation and exhalation as well as the composition of the exhaled air. The 3D headsets or VR headsets 14 would be particularly suitable for this purpose if they are fitted with a breathing tube 39. For example, the amount and speed of exhalation or the composition of the exhaled air can be recorded using the rotational speed of a rotating fan or by measuring the temperature curve or an ultrasound measurement in the exhalation direction and opposite direction or one of the other common methods. In particular, the measurement of the CO2 concentration and O2 concentration of the breathing air enables an assessment of the lung and circulatory function. For example, lung function can also be recorded at the same time using the 3D headsets. Electrical contacts for recording a brain wave curve in the form of an EEG could also be integrated into the edge of the 3D headset or into the strap for attaching the 3D headset in order to determine brain functions or O2 sensors, e.g. also on the ear to determine O2 saturation. The strap for attaching the headset can also be shaped as a hood or cap to include even more parts of the brain in the analysis.

It is also possible to use parameters collected from neck arteries and neck veins using pressure and flow sensors if, for example, the bolster 10 is designed so that it is adjacent to or in contact with the neck vessels. For example, the bolster 10 could also be concave at the top; here, too, a good direct contact with the neck vessels could be ensured by changing the pressure in the bolster 10.

All relevant sensors, such as volume sensors, pressure sensors, ultrasound sensors or flow sensors, which are used to measure the arterial pulse, flow velocity, vein diameter and vein pulse, etc., could then be accommodated in the bolster 10 or an equivalent part. By changing the position of the couch, the influence of the hydraulic pressure on the neck veins in particular can be examined.

In order to ensure perfect contact with the complexity of all electrodes and sensors, the contact should be ensured by contact measurements of the electrodes.

Audio-visual communication with the person, for example to follow instructions, is also possible, especially to eliminate a faulty electrical or mechanical contact. There should also be at least one operating switch or an operating switch controlled by voice commands.

This communication can also be used to analyze other organ systems. For example, the sense of hearing, visual acuity or the sense of smell can be assessed by transmitting changing signals via these headsets to the person 2 being examined and asking via feedback whether a certain noise or sound is still audible or a certain light signal such as writing is still visible or legible. It can also be used to monitor and measure intelligence, attention, comprehension and cognitive abilities by carrying out simple tests with the person, such as a mini-mental test or analog test methods, which the person answers via a communication medium (optical, acoustic) or a communication button.

It can also be used to assess the perception of stimuli sent by actuators, for example by acoustically communicating via the communication medium whether the intensity or quality of a certain physical signal, such as the vibration signal, or a certain heat or cold intensity can still be felt.

Point-of-care measurements of urine and blood before, during or after monitoring are also possible.

Here too, it is advantageous to integrate clinically proven products as OEM products into the diagnostic system 1. These point-of-care measurements from a tiny blood sample taken from the fingertip can be used to additionally detect or confirm liver disease, kidney disease and heart disease, such as coronary heart disease or heart failure.

With this system, a complete internal medical examination can be carried out together with the medical history taken online, without a doctor being directly involved in the examination. This is particularly beneficial in developing countries where medical care is poor. All that is needed is a chair or couch with built-in sensors and the integrated diagnostic system and, if necessary, a small point-of-care device. This system is therefore also very easy to transport to any location.

The diagnostic system 1 according to the invention makes it possible to determine the state of health of a person 2 in a particularly simple way without additional personnel. Accordingly, such a diagnostic system 1 can be used, for example, in publicly accessible places such as fitness centers, shopping malls, pharmacies, rehabilitation facilities, wellness facilities or the like, but especially in developing countries with poor medical care, where patients do not have easy access to the healthcare system. This means that people 2 can easily get a good overview of their state of health within a few minutes and discover any illnesses or disorders. Additional point of care measurements are ideal for this application, wherein personnel are helpful here. Of course, the diagnostic system 1 is also preferably configured to issue a recommendation for further action when corresponding deviations are detected, in particular whether a doctor should be consulted.

1. A diagnostic system having one or more support surfaces on which a person to be examined can be positioned, wherein electrodes are arranged on different regions of the support surface, namely on at least one leg regions, and on at least one arm regions, so that by positioning the person on the diagnostic system as intended, electrical contact is established between the electrodes and the person in order to produce an electrocardiogram, wherein these electrodes are designed at least partially as electrode pairs, wherein each electrode pair has a feed electrode and/or a measurement electrode, wherein the feed electrode is arranged distally relative to the measurement electrode, and wherein a device for feeding an electric current between the feed electrodes is provided and wherein the diagnostic system is configured to measure a voltage drop between the measurement electrodes in order to be able to determine impedances of body regions of a person located on the diagnostic system, wherein at least one electrode is arranged at an area which can be pressed with variable pressure against the person using the diagnostic system, wherein the area is designed as a bladder which can be filled with a fluid, wherein a pressure of the fluid in the bladder can be varied.

2. The diagnostic system according to claim 1, wherein the diagnostic system is configured to switch individual electrodes or electrode pairs in pairs in such a way that an electric current, in particular an alternating current, is fed in with two feed electrodes and a voltage drop between the associated measurement electrodes can be measured.

3. The diagnostic system according to claim 1, wherein a measurement path is designed in such a way that only a part of the measurement path has electric current flowing through it and the other part of the measurement path is designed as an electrical conductor.

4. The diagnostic system according to claim 1, wherein more than one current source is provided, wherein these are preferably electrically isolated from one another and connectable to the feed electrodes.

5. The diagnostic system according to claim 1, wherein the diagnostic system is configured for feeding alternating electrical currents with the individual feed electrodes with different frequencies, in particular with frequencies of 1 Hz to 1 MHz, in particular 5 kHz to 500 KHz.

6. The diagnostic system according to claim 1, wherein switches are provided with which the individual feed electrodes can be switched directly adjacent to coupling points, in particular less than 3 cm away from coupling points, at which coupling points a person located on the diagnostic system can make electrical contact with the electrodes.

7. The diagnostic system according to claim 1, wherein an electrode contact measurement is provided, with which those electrodes or electrode pairs can be identified which have the lowest contact resistance to a person to be examined, wherein the diagnostic system is configured to activate those electrode pairs which have the lowest contact resistance, in particular by switches which are arranged close to the coupling points.

8. The diagnostic system according to claim 7, wherein the electrode contact measurement is configured to apply an electrical voltage to individual electrodes in order to achieve a current flow via a body located on the diagnostic system, so that a contact resistance can be determined on the basis of a level of the current.

9. The diagnostic system according to claim 7, wherein the electrode contact measurement can be switched off, in particular close to the coupling point of the person.

10. The diagnostic system according to claim 1, wherein matched current sources are connected to the feed electrodes.

11. The diagnostic system according to claim 10, wherein one of the matched current sources is designed as a current source and the other as a current sink.

12. The diagnostic system according to claim 1, wherein the diagnostic system is designed to perform the regulation and matching of the current sources taking into account the common-mode voltage of the person, which is measured via a right-leg-drive electrode.

13. The diagnostic system according to claim 1, wherein the impedance measurements are carried out as plethysmographic measurements and as multi-frequency measurements, in particular as segmental impedance measurements.

14. The diagnostic system according to claim 13, wherein the plethysmographic measurement is performed as a pulse-synchronous measurement.

15. The diagnostic system according to claim 13, wherein a segmental and/or whole-body plethysmographic measurement is provided, which is designed in such a way that only a part of a measurement path has an electric current flowing through it and another part of the measurement path is designed as an electrical conductor.

16. The diagnostic system according to claim 15, wherein the whole-body plethysmographic measurement is carried out in such a way that the alternating current is fed to at least one arm and one leg and that the impedance measurement is carried out between the arm and leg of the impedance feed.

17. The diagnostic system according to claim 15, wherein the segmental and/or whole-body plethysmographic measurement is carried out in such a way that an R-wave detector detects the R-wave, with the aid of which the impedance plethysmographic signals of more than one signal are then superimposed, and wherein a template of the impedance plethysmographic signal is generated and wherein the cardiac performance, the physical performance, the pulse wave or volume wave are calculated.

18. The diagnostic system according to claim 1, wherein a preferably elastically shaped tensioning strap is present, which is mechanically and electrically connected at a seam to a bearing surface and/or support surface, and which establishes the connection to electrodes at electrode positions Iᵣ, Mᵣ, Aᵣ, E, A, I and to chest wall electrodes and possibly further electrodes.

19. The diagnostic system according to claim 1, wherein a multi-channel ECG is present.

20. The diagnostic system according to claim 19, wherein at least three ECG leads are arranged between two electrodes each as orthogonally as possible on the support surface, one approximately centrally, approximately corresponding to the longitudinal axis in the upper region of the support surface, the second approximately in the central longitudinal region of the support surface, approximately transversely to the longitudinal direction of the support surface, and the third approximately diagonally in the upper region of the support surface, preferably directed to approximately 10 h 30, alternatively to approximately 1 h 30, corresponding to the hour hand of a clock.

21. The diagnostic system according to claim 1, wherein electrodes are present at electrode positions E, A, S and I for determining EASI leads.

22. The diagnostic system according to claim 21, wherein the electrode for the lead S according to EASI is attached separately in the head, neck or upper body region.

23. The diagnostic system according to claim 22, wherein the electrodes, at least at electrode positions E, A and I according to EASI, are present on a common strap.

24. The diagnostic system according to claim 1, wherein the electrode for the lead S according to EASI is present on a band, preferably on VR headsets, in the head, neck or upper body region.

25. The diagnostic system according to claim 1, wherein the diagnostic system is designed to use ECG signals derived from the coupling point of the back of the person with the support surface, in particular from the electrode positions Iᵣ, Aᵣ, Mᵣ, Nᵣ, Fᵣ, to reconstruct conventional ECG leads according to Wilson, and/or Einthoven and/or Goldberger, Nehb and Frank.

26. The diagnostic system according to claim 1, wherein the diagnostic system is designed to calculate the leads according to Wilson and/or Goldberger, Nehb and Frank with the aid of regression equations using the amplitudes of the individual segments of the ECG, obtained from the electrode positions of the support surface, in particular Iᵣ, Aᵣ, Mᵣ, Nᵣ, Fr, namely the PR segment, QRS complex, the ST-T segment and the T wave, in particular with the aid of linear regression equations and/or non-linear equations and/or neural networks and/or other artificial intelligence methods.

27. The diagnostic system according to claim 1, wherein the diagnostic system is configured so that the leads according to Wilson, and/or Einthoven and/or Goldberger, Nehb and Frank are calculated with the aid of the regression equations V derived=a(Aᵣ−Iᵣ)+b(Mᵣ−Nᵣ)+c (Aᵣ−Nᵣ)+d, wherein the coefficients a, b, c, d are linear or non-linear coefficients.

28. The diagnostic system according to claim 1, wherein the support surface is preferably deformable, in particular elastically deformable, preferably as an elastomer, and has a plurality of at least partially convex and/or concave, preferably elastic deformations which support the electrodes.

29. The diagnostic system according to claim 1, wherein at least one electrode is arranged at the neck region on an elastic bolster, which in particular comprises an elastomer.

30. The diagnostic system according to claim 1, wherein at least one electrode, in particular a leg electrode on the legs and an arm electrode on the arms, is concave in order to be able to pick up a corresponding region of the human body, in particular a leg region and/or arm region and/or knee region and/or neck region.

31. The diagnostic system according to claim 1, wherein at least one arm electrode and at least one leg electrode are provided several times for the same extremity, wherein these are not arranged in a line along an extremity beam as seen from the leg base, but are laterally displaced with respect to one another, so that only one electrode can pick up the extremity in each case and the others come to lie next to it, in order to be able to adapt the diagnostic system to persons of different sizes.

32. The diagnostic system according to claim 1, wherein at least two electrodes can be moved relative to one another, in particular the electrodes on the leg regions relative to the electrodes in the upper body region and/or the electrodes on the arm regions relative to the electrodes on the upper body region, in order to be able to adapt the diagnostic system to persons of different sizes.

33. The diagnostic system according to claim 1, wherein several electrodes are arranged at the leg regions, the arm regions, the knee regions, the neck region and/or the upper body region, wherein the diagnostic system is configured to activate one or more of these several electrodes depending on a size of the person to be examined, in order to ensure a correct electrode position irrespective of a size of the person to be examined.

34. The diagnostic system according to claim 1, wherein at least three electrodes are provided at approximately the same height in the upper body region, wherein one electrode is arranged approximately centrally and the two further electrodes being arranged approximately symmetrically laterally thereto.

35. The diagnostic system according to claim 1, wherein the support surface, which contacts a person positioned on the diagnostic system, has a bearing surface at least in a region of the electrodes, which is formed in particular from a durable pressure-resistant material.

36. The diagnostic system according to claim 35, wherein a fluid is arranged between the bearing surface and the support surface.

37. The diagnostic system according to claim 35, wherein electrical lines to the individual electrodes and/or sensors, such as in particular pressure sensors, light sensors, sound sensors, acceleration sensors and the like, are arranged between the bearing surface and the support surface.

38. The diagnostic system according to claim 1, wherein a device for changing a pressure in the fluid is provided, wherein further a pressure sensor for determining a pressure in the fluid is provided.

39. The diagnostic system according to claim 1, wherein actuators are provided with which, in particular in regions of the electrodes, mechanical or electrical forces and/or movements, in particular pressure, vibrations, alternating current, direct current, temperature, light, can be applied to parts of the body of a person located on the diagnostic system.

40. The diagnostic system according to claim 1, wherein the support surface and bearing surface are U-shaped in some regions, in particular in an extremity region, and, in particular in a region of the electrodes, are designed to build up a precisely defined pressure, in particular continuously, in order to determine a pulse and/or a blood pressure of a person positioned on the diagnostic system oscillometrically and/or auscultatorily and/or by vascular unloading.

41. The diagnostic system according to claim 1, wherein at least one upper arm cuff and/or one leg cuff and/or one pulse oximeter are provided.

42. The diagnostic system according to claim 1, wherein the diagnostic system has sensors with which a mechanical response of the parts of the body of a person located on the diagnostic system to applied forces and/or movements can be detected in order to be able to assess mechanical stiffnesses of individual body regions and/or sensitivities of the person, in particular as a function of a frequency and amplitude with which a force and/or a movement are applied.

43. The diagnostic system according to claim 1, wherein the diagnostic system is designed as a couch, in particular as a tilting couch, wherein a tilting angle can be detected.

44. The diagnostic system according to claim 1, wherein the support surface and the bearing surface are arranged together as a mat, in particular as a continuous mat.

45. The diagnostic system according to claim 1, wherein one or more cameras, and/or a thermal imaging camera and/or infrared camera, and/or time-of-flight sensors are provided, with which a person located on the support surface can be detected three-dimensionally and/or with which physical properties, in particular dimensions, color and/or temperature of a person being examined can be determined.

46. The diagnostic system according to claim 1, wherein the diagnostic system is configured to combine data that is acquired via electrodes with data that is acquired without contact, in particular with data that is acquired via a camera, and equations or sets of equations are created from the combined data to calculate biological data.

47. The diagnostic system according to claim 1, wherein a chest strap is provided, which has chest wall electrodes on the inside and chest strap outer electrodes on the outside, which correspond to contact electrodes on the support surface, so that electrical signals from the chest wall electrodes can be transmitted via the contact electrodes on the support surface.

48. The diagnostic system according to claim 1, wherein devices for generating physical effects, such as mechanical effects such as vibration or temperature, such as heat and cold, or electricity, are provided at different positions in order to be able to determine reactions of a person located at the diagnostic system to applied physical changes.

49. The diagnostic system according to claim 1, wherein an optical (screen, virtual reality headset) and/or acoustic communication medium, which can be formed, for example, as a microphone and/or headphones, is provided in a field of vision and/or communication region of a person positioned on the diagnostic system, which is connected to the diagnostic system via a data connection, so that data relating in particular to a state of health of the person can be displayed on the communication medium and/or communication with the person is possible.

50. The diagnostic system according to claim 49, wherein the communication medium is designed in such a way that information, particularly from the regions of relaxation, biofeedback, knowledge, especially medical knowledge, and advertising, especially user-specific advertising, can be conveyed.

51. The diagnostic system according to claim 49, wherein the communication medium is virtual reality headset and has sensors for recording health-related data of the person, in particular sensors for recording physical variables such as electricity, light, temperature, sound.

52. The diagnostic system according to claim 1, wherein the communication medium is virtual reality headset equipped with a breathing tube which is equipped for measuring flow velocity, breathing volume, breathing pressure and/or for gas analysis, especially for CO2, O2 and volatile gases.

53. The diagnostic system according to claim 1, wherein it is configured to use artificial intelligence, for example deep convolutional neural nets, genetic algorithms and/or other methods, to evaluate the data, in particular for impedance plethysmographic and impedance spectroscopic data analysis.

54. The diagnostic system according to claim 1, wherein a bidirectionally communicating online expert system for the diagnosis of numerous diseases is present, with which the diagnostic system is connected via a data connection, in particular via the Internet, and/or which makes further suggestions regarding diagnosis and therapy.

55. The diagnostic system according to claim 1, wherein it is configured to diagnose at least one of myocardial infarction, coronary heart disease, pulmonary embolism, right heart overload, valvular heart disease, arrhythmia, atrial fibrillation and risk of atrial fibrillation, heart rate variability, autonomic nervous system, sympathetic nervous system, vagus, baroreceptor sensitivity, blood pressure, cardiac performance, diagnosis of heart failure, hydration status, muscle mass, muscle strength, biological muscle age, fat mass, abdominal fat to assess metabolic risk, thrombosis, Lymphoedema, pulse wave transit time, lung function, respiratory gases, oxygen saturation, continuous beat-to-beat blood pressure, aortic stiffness, central aortic pressure, ankle-brachial index, circulatory disorders of the legs, central blood pressure, carotid artery disease, atherosclerosis, compaction of the liver, kidney function, medication dosage, e.g. for cytostatics or narcotics, inflammation, diabetic foot, tumors, brain function disorders such as epilepsy, loss of brain function, polyneuropathy, visual examination, hearing examination, metabolic diseases such as diabetes, osteoporosis or
bone metabolism disorders and disorders of all internal organs, malignancies, follow-up observations and other diagnoses.

56. The diagnostic system according to claim 1, wherein mechanical joints, in particular hinge joints with actuators, for moving the joints as one or more therapy devices are present in connection with the support surface where the joints, in particular hip joints, knee joints, ankle joints, shoulder joints, elbow joints, wrist joints of the examined body are positioned.

57. The diagnostic system according to claim 56, wherein the actuators are designed as physiotherapy devices, massage devices and/or vibration devices and/or heat-cold application devices and/or application devices for electric current.

58. The diagnostic system according to claim 1, wherein at least one, preferably each, electrode is arranged at an area which can be pressed with variable pressure against a human body or a person using the diagnostic system, wherein the area is designed in particular as a bladder which can be filled with a fluid, wherein a pressure of the fluid in the bladder can be changed, preferably by a pump.

59. The diagnostic system according to claim 1, wherein an arrangement of at least three, preferably six, deformable areas to be subjected to a controllable pressure is present, wherein at least one area which is located centrally with respect to the human body, i.e. approx. in the center of the body, is equipped with at least three electrodes arranged substantially horizontally in relation to the body axis, and wherein at least two, preferably five, peripherally located areas to be subjected to a preferably controllable pressure are present, which are at least partially equipped with at least two electrodes which have an at least approximately vertical orientation in relation to the body axis.

60. The diagnostic system according to claim 59, wherein the centrally located area is located predominantly in front of the examined human body and the other areas are located behind the human body.

61. The diagnostic system according to claim 59, wherein the areas have a bladder or a chamber with variable pressure which is filled with a fluid.

62. The diagnostic system according to claim 61, wherein the fluid is a gas, for example air or CO2.

63. The diagnostic system according to claim 61, wherein the deformation of the areas can be carried out by increasing the pressure in the fluid or by hydraulic and/or mechanical devices, for example a plunger.

64. The diagnostic system according to claim 61, wherein the electrodes consist of a deformable conductive fabric tape.

65. The diagnostic system according to claim 61, wherein at least one controlled pressure pump, preferably hydraulic, is provided for changing the pressure in the bladders.

66. The diagnostic system according to claim 61, wherein the bubbles are filled with a fluid, preferably water, and can serve as a fluid feed for further examinations and therapies.

67. The diagnostic system according to claim 61, wherein the bubbles are shaped in such a way that they serve for the propagation of waves, e.g. light, temperature, sound—in particular also ultrasound—and thus for diagnoses and therapies.

68. The diagnostic system according to claim 61, wherein the e.g. U-shaped support surface for the arms and legs is subjected to continuously or incrementally variable pressures, and thus e.g. the blood pressure is measured oscillometrically and/or auscultatorily, possibly also additionally continuously, e.g. by vascular unloading.

69. The diagnostic system according to claim 1, wherein a strap, preferably a safety strap, preferably with a snap-on device, is present, on which at least one, preferably at least three, fluid-filled deformable and/or displaceable areas equipped with electrodes are present, wherein the at least one area, preferably three areas, is arranged approximately horizontally in relation to the body axis and preferably carries three electrodes arranged horizontally in relation to the body axis.

70. The diagnostic system according to claim 69, wherein a tensioning device is provided for the safety strap.

71. The diagnostic system according to claim 69, wherein the areas or bladder with the horizontal electrodes is slidably attached to the strap, and the electrodes E and/or Mᵣ can be positioned centrally in relation to the positioned person even if the circumference of the body is different.

72. The diagnostic system according to claim 1, wherein a bolster is movably, in particular displaceably, attached to the support surface.

73. The diagnostic system according to claim 1, wherein a bolster is U-shaped.

74. The diagnostic system according to claim 1, wherein the ECG leads according to Einthofen, Wilson and Goldberger are present and/or by ECG leads between the electrode located on the left on the chest strap or on the support surface at electrode positions A, Aᵣ and the electrode located on the right on the chest strap or on the support surface at electrode positions I, Iᵣ, furthermore between the electrode located on the left of the chest strap or on the support surface at electrode positions A, Aᵣ and the neck electrode at electrode position S or N, and finally between the electrode located in the middle of the chest strap or on the support surface at electrode position E, Eᵣ or also M and the neck electrode at electrode position S or also N.

75. The diagnostic system according to claim 74, wherein the ECG leads for the individual segments of the ECG curve, e.g. for the segments P-wave, PQ-wave, R-wave, ST-wave and T-wave, are reconstructed by separate sets of equations.

76. The diagnostic system according to claim 1, wherein a non-contact measurement of the examined person, e.g. by optical methods and/or time-of-flight methods, is integrated.

77. The diagnostic system according to claim 1, wherein the thermal radiation of the person being examined is measured, for example by a thermal imaging camera.

78. The diagnostic system according to claim 1, wherein it is designed to be used for the diagnosis and/or therapy of diseases of the central nervous system, in particular also of the autonomic and sensitive nervous system, the sensory organs, in particular the eyes, the ears, furthermore the vessels, in particular the vessels of the neck and peripheral vessels, the lungs, the heart, the liver, the kidneys, the bones, the muscles, the joints, the peripheral nervous system.

79. The diagnostic system according to claim 1, wherein the diagnostic system is configured to record the recording of the biological signals before, during and/or after a change of state, such as before and after tilting maneuvers, change of a respiratory state and thus to use the change in the measured values of the circulation, the body fluids and the vascular properties triggered by the maneuver for the diagnosis.

80. The diagnostic system according to claim 1, wherein the electrodes are arranged displaceably or rotatably at arm regions and/or the electrodes are arranged rotatably or displaceably at leg regions, wherein preferably the axes of rotation for the electrodes in the arm region run approximately perpendicular to the support surface and for the electrodes in the leg region run approximately parallel to the support surface when the person is located on the diagnostic system.

81. The diagnostic system according to claim 1, wherein the electrodes are designed for contactless detection of measured values for an ECG, in particular as capacitive electrodes.

82. The diagnostic system according to claim 1, wherein the diagnostic system is configured for determining the lean body mass and/or the muscle mass and for correcting a kidney function based on the lean body mass and/or the muscle mass using the electrical measured values determined with the electrodes and/or measured values determined without contact, in particular measured values determined with a camera.

83. The diagnostic system according to claim 1, wherein one, preferably two, microphones are provided in a chest region, which microphones can preferably be pressed against a human body by areas with variable pressure, in particular bubbles filled with a fluid, in order to detect lung sounds.
